(19) United States
(12) Patent Application Publication
(10) Pub. No.: US 2010/0305021 A1
(43) Pub. Date: Dec. 2, 2010

(54) PERFUME DELIVERY SYSTEMS FOR CONSUMER GOODS

(76) Inventor: Robert Richard Dykstra, West Chester, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/852,828

(22) Filed: Aug. 9, 2010

Related U.S. Application Data
(63) Continuation of application No. 11/801,260, filed on May 9, 2007.
(60) Provisional application No. 60/922,673, filed on Apr. 10, 2007.
(60) Provisional application No. 60/802,707, filed on May 23, 2006.

(51) Int. Cl. A61K 8/11; A61Q 13/00

(52) U.S. Cl. 512/4

(57) ABSTRACT

The present invention relates to perfume delivery systems, products comprising such systems and the use of same. Unfortunately current perfume delivery systems do not always provide the most preferred or ideal sensory experience. Thus there is a need for one or more perfume delivery systems that obviate the short comings of the current perfume delivery technologies. The systems of the present invention meet the aforementioned need as they employ symbiotic combinations.

### CROSS-REFERENCES TO RELATED APPLICATIONS

This application claims priority under 35 U.S.C. §120 to U.S. application Ser. No. 11/801,260, filed May 9, 2007, which in turn claims priority under 35 U.S.C. §119(e) to U.S. Provisional Application Ser. No. 60/922,673 filed Apr. 10, 2007 and U.S. Provisional Application Ser. No. 60/802,707 filed May 23, 2006.

### BACKGROUND OF THE INVENTION

Products such as consumer products are typically designed and/or formulated to include a perfume system. The consumer who selects and uses such a product typically experiences at least three product moments of truth. The first moment of truth is typically at the point of purchase, the second moment of truth typically begins with the product's application and use, and the third moment of truth typically begins immediately after the product's application and use. Unfortunately, current perfume delivery systems do not always provide the most preferred or ideal sensory experience during such moments of truth. Thus, there is a need for one or more perfume delivery systems that obviate the short comings of the current perfume delivery technologies. The perfume systems of the present invention meet the aforementioned need.

### SUMMARY OF THE INVENTION

The present invention relates to perfume delivery systems, products comprising such systems and the use of same.

### DETAILED DESCRIPTION OF THE INVENTION

### Definitions

As used herein “FMOT” means first moment of truth.

As used herein “SMOT” means second moment of truth.

As used herein “TMOT” means third moment of truth.

As used herein “PRM” means perfume raw material.

As used herein “perfume delivery system” encompasses a single perfume delivery technology and/or combinations of perfume delivery technologies.

As used herein, the terms “perfume system” and “perfume delivery system” are synonymous.

As used herein “consumer products” includes, unless otherwise indicated, articles, baby care, beauty care, fabric & home care, family care, feminine care, health care, snack and/or beverage products or devices intended to be used or consumed in the form in which it is sold, and is not intended for subsequent commercial manufacture or modification. Such products include but are not limited to diapers, bibs, wipes; products for and/or methods relating to treating hair (human, dog, and/or cat), including bleaching, coloring, dyeing, conditioning, shampooing, styling; deodorants and antiperspirants; personal cleansing products, including cleansers, moisturizing cleansers, and combinations thereof; cosmetics; skin care including application of creams, lotions, mousses, masks, exfoliating compositions, peels, and combinations thereof; hair removal products, including device-assisted hair removal products; shaving products; and other topically applied products for consumer use; products for and/or methods relating to treating fabrics, hard surfaces and any other surfaces in the area of fabric and home care, including: air care, car care, dishwashing, fabric conditioning (including softening), laundry detergency, laundry and rinse additive and/or care, hard surface cleaning and/or treatment, and other cleaning for consumer or institutional use; products and/or methods relating to bath tissue, facial tissue, paper handkerchiefs, and/or paper towels; tampons, feminine napkins; products and/or methods relating to oral care including toothpastes, tooth gels, tooth rinses, denture adhesives, tooth whitening; over-the-counter health care including cough and cold remedies, pain relievers, pet health and nutrition, and water purification; processed food products intended primarily for consumption between customary meals or as a meal accompaniment (non-limiting examples include potato chips, tortilla chips, popcorn, pretzels, corn chips, cereal bars, vegetable chips or crisps, snack mixes, party mixes, multigrain chips, snack crackers, cheese snacks, pork rinds, corn snacks, pellet snacks, extruded snacks and bagel chips); and coffee and cleaning and/or treatment compositions.

As used herein, the term “cleaning and/or treatment composition” includes, unless otherwise indicated, tablet, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, cleaning bars, mouthwashes, denture cleaners, car or carpet shampoos, bathroom cleaners; hair shampoos and hair-rinses; shower gels and foam baths and metal cleaners; as well as cleaning auxiliaries such as bleach additives and “stain-stick” or pre-treat types substrate-laden products such as dryer added sheets, dry and wetted wipes and pads, non-woven substrates, and sponges; as well as sprays and mists.

As used herein, the term “fabric care composition” includes, unless otherwise indicated, fabric softening compositions, fabric enhancing compositions, fabric freshening compositions and combinations thereof.

As used herein, the term “solid” includes granular, powder, bar and tablet product forms.

As used herein, the term “situs” includes paper products, fabrics, garments, hard surfaces, hair and skin.

As used herein, the articles a and an when used in a claim, are understood to mean one or more of what is claimed or described.

For purposes of the present invention, and unless indicated otherwise, the terms “monomer-assisted delivery” and “material-assisted delivery” are within the scope of the term “molecule-assisted delivery.”

For purposes of the present invention, and unless indicated otherwise, the terms “molecule” and “non-polymer” are within the scope of the term “monomer.”

For purposes of the present invention and unless indicated otherwise, the terms “perfume nanocapsule” and “microcapsule” are within the scope of the term “perfume microcapsule.”

Unless otherwise noted, all component or composition levels are in reference to the active level of that component or composition, and are exclusive of impurities, for example, residual solvents or by-products, which may be present in commercially available sources.

All percentages and ratios are calculated by weight unless otherwise indicated. All percentages and ratios are calculated based on the total packaged product, which includes the product and product matrix composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

### Perfume Delivery Systems

The consumer who selects and uses such a perfumed product makes critical decisions as to how satisfied he or she is with the product at multiple touch points in the product usage profile. Although numerous touch points are known, Applicants have found that they can be advantageously grouped and expressed as three product moments of truth that are experienced by the typically consumer. The FMOT is typically at the point of purchase, the SMOT typically begins with the product's application and use, and the TMOT typically begins immediately after the product's application and use. Applicants have recognized that a consumer's FMOT is negatively impacted because the product packaging inhibits the sensory experience; for example, product packaging may make the product difficult to open or, when open, exposes a product that can spill. In addition, formulation ingredients can suppress and/or distort neat product odor. Furthermore Applicants have recognized that the consumer's SMOT is negatively impacted as volatile PRMs are lost during product storage, resulting in reduced bloom during use. Compensating for these aforementioned deficiencies by adding high perfume levels for the TMOT can distort in-use scent experience, such that the perfume bloom can be too harsh or strong, and/or the perfume character can become less preferred. Also, Applicants have recognized that a consumer's FMOT is negatively impacted as perfume releases from the treated situs, inter alia a dry fabric over long period of time requires perfume levels in product that would distort the scent experience during the first and second moments of truth. Furthermore, addition of high perfume levels for SMOT & TMOT can distort neat product odor, and still not result in sufficient perfume deposition through the wash. In addition, perfume evaporation that occurs during drying can result in lower perfume levels on fabric; and/or the perfume remaining on dry fabric may provide initial dry fabric odor benefit but such perfume can dissipate too quickly to provide sufficient scent longevity benefits. Furthermore, perfume that is present on fabric may release too slowly from the fabric. As mentioned, the same can be the case with perfume delivery to and release from other situs such as hair and skin. The ability to notice the release of perfume can be impacted by a variety of factors such as hair length, clothing worn over skin, situs wash frequency, and the like. Variable deposition and release during the wash or rinse or application can be another deficiency that can negatively impact the scent experience during the different moments of truth. The number of treatments or applications can also have an impact, for example, wherein a different intensity and/or character is achieved after the first wash compared to after subsequent washes of a particular situs. Furthermore, perfume intensity and/or character may be perceived differently on wet situs compared to dry situs that is treated with perfume-containing products. Without wishing to be bound by theory, in addition to loss of perfume by evaporation during drying, perfume can be made less available at certain touch points by being carried into or partitioning into the situs, such as cotton fibers, hair, skin, and the like. Situs moisture level can also serve to alter the release profile or release rate of perfume.

Finally, Applicants recognized that solutions to the problems that are associated with one or two moments of truth can be insufficient to resolve the problems associated with the remaining moment(s) of truth or negatively impact the other moment(s) of truth

The following perfume delivery technologies (PDTs) also known as perfume delivery systems may be used in any combination in any type of consumer product:

Polymer Assisted Delivery (PAD): This perfume delivery technology uses polymeric materials to deliver perfume materials. Classical coacervation, water soluble or partly soluble to insoluble charged or neutral polymers, liquid crystals, hot melts, hydrogels, perfumed plastics, microcapsules, nano- and micro-latexes, polymeric film formers, and polymeric absorbents, polymeric adsorbents, etc. are some examples. PAD includes but is not limited to:
Matrix Systems: The fragrance is dissolved or dispersed in a polymer matrix or particle. Perfumes, for example, may be 1) dispersed into the polymer prior to formulating into the product or 2) added separately from the polymer during or after formulation of the product. Diffusion of perfume from the polymer is a common trigger that allows or increases the rate of perfume release from a polymeric matrix system that is deposited or applied to the desired surface (situs), although many other triggers are know that may control perfume release. Absorption and/or adsorption into or onto polymeric particles, films, solutions, and the like are aspects of this technology. Nano- or micro-particles composed of organic materials (e.g., latexes) are examples. Suitable particles include a wide range of materials including, but not limited to polyacetal, polyacrylate, polyacrylic, polyacrylonitrile, polyamide, polyaryletherketone, polybutadiene, polybutylene, polybutylene terephthalate, polychloroprene, poly ethylene, polyethylene terephthalate, polycyclohexylene dimethylene terephthalate, polycarbonate, polychloroprene, polyhydroxyalkanoate, polyketone, polyester, polyethylene, polyetherimide, polyethersulfone, polyethylenechlorinates, polyimide, polyisoprene, polylactic acid, polymethylpentene, polyphenylene oxide, polyphenylene sulfide, polyphthalamide, polypropylene, polystyrene, polysulfone, polyvinyl acetate, polyvinyl chloride, as well as polymers or copolymers based on acrylonitrile-butadiene, cellulose acetate, ethylene-vinyl acetate, ethylene vinyl alcohol, styrene-butadiene, vinyl acetate-ethylene, and mixtures thereof.

“Standard” systems refer to those that are “pre-loaded” with the intent of keeping the pre-loaded perfume associated with the polymer until the moment or moments of perfume release. Such polymers may also suppress the neat product odor and provide a bloom and/or longevity benefit depending on the rate of perfume release. One challenge with such systems is to achieve the ideal balance between 1) in-product stability (keeping perfume inside carrier until you need it) and 2) timely release (during use or from dry situs). Achieving such stability is particularly important during in-product storage and product aging. This challenge is particularly apparent for aqueous-based, surfactant-containing products, such as heavy duty liquid laundry detergents. Many “Standard” matrix systems available effectively become “Equilibrium” systems when formulated into aqueous-based products. One may select an “Equilibrium” system or a Reservoir system, which has acceptable in-product diffusion stability and available triggers for release (e.g., friction). “Equilibrium” systems are those in which the perfume and polymer may be added separately to the product, and the equilibrium interaction between perfume and polymer leads to a benefit at one or more consumer touch points (versus a free perfume control that has no polymer-assisted delivery technology). The polymer may also be pre-loaded with perfume; however, part or all of the perfume may diffuse during in-product storage reaching an equilibrium that includes having desired perfume raw materials (PRMs) associated with the polymer. The polymer then carries the perfume to the surface, and release is typically via perfume diffusion. The use of such equilibrium system polymers has the potential to decrease the neat product odor intensity of the neat product (usually more so in the case of pre-loaded standard system). Deposition of such polymers may serve to “flatten” the release profile and provide increased longevity. As indicated above, such longevity would be achieved by suppressing the initial intensity and may enable the formulator to use more high impact or low odor detection threshold (ODT) or low Kovats Index (KI) PRMs to achieve FMOT benefits without initial intensity that is too strong or distorted. It is important that perfume release occurs within the time frame of the application to impact the desired consumer touch point or touch points. Suitable micro-particles and micro-latexes as well as methods of making same may be found in USPA 2005/0003980 A1. Matrix systems also include hot melt adhesives and perfume plastics. In addition, hydrophobically modified polysaccharides may be formulated into the perfumed product to increase perfume deposition and/or modify perfume release. All such matrix systems, including for example polysaccarides and nanolatexes may be combined with other PDTs, including other PAD systems such as PAD reservoir systems in the form of a perfume microcapsule (PMC). Polymer Assisted Delivery (PAD) matrix systems may include those described in the following references: US Patent Applications 2004/0110648 A1; 2004/0092414 A1; 2004/0091445 A1 and 2004/0087476 A1; and U.S. Pat. Nos. 6,531,444; 6,024,943; 6,042,792; 6,051,540; 4,540,721 and 4,973,422.

Silicones are also examples of polymers that may be used as PDT, and can provide perfume benefits in a manner similar to the polymer-assisted delivery “matrix system”. Such a PDT is referred to as silicone-assisted delivery (SAD). One may pre-load silicones with perfume, or use them as an equilibrium system as described for PAD. Suitable silicones as well as making same may be found in WO 2005/102261; USPA 20050124530A1; USPA 20050143282A1; and WO 2003/015736. Functionalized silicones may also be used as described in USPA 2006/003913 A1. Examples of silicones include polydimethylsiloxane and polyalkyldimethylsiloxanes. Other examples include those with amine functionality, which may be used to provide benefits associated with amine-assisted delivery (AAD) and/or polymer-assisted delivery (PAD) and/or amine-reaction products (ARP). Other such examples may be found in U.S. Pat. No. 4,911,852; USPA 2004/0058845 A1; USPA 2004/0092425 A1 and USPA 2005/0003980 A1.

Reservoir Systems: Reservoir systems are also known as a core-shell type technology, or one in which the fragrance is surrounded by a perfume release controlling membrane, which may serve as a protective shell. The material inside the microcapsule is referred to as the core, internal phase, or fill, whereas the wall is sometimes called a shell, coating, or membrane. Microparticles or pressure sensitive capsules or microcapsules are examples of this technology. Microcapsules of the current invention are formed by a variety of procedures that include, but are not limited to, coating, extrusion, spray-drying, interfacial, in-situ and matrix polymerization. The possible shell materials vary widely in their stability toward water. Among the most stable are polyoxymethyleneurea (PMU)-based materials, which may hold certain PRMs for even long periods of time in aqueous solution (or product). Such systems include but are not limited to urea-formaldehyde and/or melamine-formaldehyde. Gelatin-based microcapsules may be prepared so that they dissolve quickly or slowly in water, depending for example on the degree of cross-linking. Many other capsule wall materials are available and vary in the degree of perfume diffusion stability observed. Without wishing to be bound by theory, the rate of release of perfume from a capsule, for example, once deposited on a surface is typically in reverse order of in-product perfume diffusion stability. As such, urea-formaldehyde and melamine-formaldehyde microcapsules for example, typically require a release mechanism other than, or in addition to, diffusion for release, such as mechanical force (e.g., friction, pressure, shear stress) that serves to break the capsule and increase the rate of perfume (fragrance) release. Other triggers include melting, dissolution, hydrolysis or other chemical reaction, electromagnetic radiation, and the like. The use of pre-loaded microcapsules requires the proper ratio of in-product stability and in-use and/or on-surface (on-situs) release, as well as proper selection of PRMs. Microcapsules that are based on urea-formaldehyde and/or melamine-formaldehyde are relatively stable, especially in near neutral aqueous-based solutions. These materials may require a friction trigger which may not be applicable to all product applications. Other microcapsule materials (e.g., gelatin) may be unstable in aqueous-based products and may even provide reduced benefit (versus free perfume control) when in-product aged. Scratch and sniff technologies are yet another example of PAD. Perfume microcapsules (PMC) may include those described in the following references: US Patent Applications: 2003/0125222 A1; 2003/215417 A1; 2003/216488 A1; 2003/158344 A1; 2003/165692 A1; 2004/071742 A1; 2004/071746 A1; 2004/072719 A1; 2004/072720 A1; 2006/0039934 A1; 2003/203829 A1; 2003/195133 A1; 2004/087477 A1; 2004/0106536 A1; and U.S. Pat. Nos. 6,645,479 B1; 6,200,949 B1; 4,882,220; 4,917,920; 4,514,461; 6,106,875 and 4,234,627, 3,594,328 and US RE 32713.
Molecule-Assisted Delivery (MAD): Non-polymer materials or molecules may also serve to improve the delivery of perfume. Without wishing to be bound by theory, perfume may non-covalently interact with organic materials, resulting in altered deposition and/or release. Non-limiting examples of such organic materials include but are not limited to hydrophobic materials such as organic oils, waxes, mineral oils, petrolatum, fatty acids or esters, sugars, surfactants, liposomes and even other perfume raw material (perfume oils), as well as natural oils, including body and/or other soils. Perfume fixatives are yet another example. In one aspect, non-polymeric materials or molecules have a CLogP greater than about 2. Molecule-Assisted Delivery (MAD) may also include those described in U.S. Pat. No. 7,119,060 and U.S. Pat. No. 5,506,201.
Fiber-Assisted Delivery (FAD): The choice or use of a situs itself may serve to improve the delivery of perfume. In fact, the situs itself may be a perfume delivery technology. For example, different fabric types such as cotton or polyester will have different properties with respect to ability to attract and/or retain and/or release perfume. The amount of perfume deposited on or in fibers may be altered by the choice of fiber, and also by the history or treatment of the fiber, as well as by any fiber coatings or treatments. Fibers may be woven and non-woven as well as natural or synthetic. Natural fibers include those produced by plants, animals, and geological processes, and include but are not limited to cellulose materials such as cotton, linen, hemp jute, flax, ramie, and sisal, and fibers used to manufacture paper and cloth. Fiber-Assisted Delivery may consist of the use of wood fiber, such as thermomechanical pulp and bleached or unbleached kraft or sulfite pulps. Animal fibers consist largely of particular proteins, such as silk, sinew, catgut and hair (including wool). Polymer fibers based on synthetic chemicals include but are not limited to polyamide nylon, PET or PBT polyester, phenol-formaldehyde (PF), polyvinyl alcohol fiber (PVOH), polyvinyl chloride fiber (PVC), polyolefins (PP and PE), and acrylic polymers. All such fibers may be pre-loaded with a perfume, and then added to a product that may or may not contain free perfume and/or one or more perfume delivery technologies. In one aspect, the fibers may be added to a product prior to being loaded with a perfume, and then loaded with a perfume by adding a perfume that may diffuse into the fiber, to the product. Without wishing to be bound by theory, the perfume may absorb onto or be adsorbed into the fiber, for example, during product storage, and then be released at one or more moments of truth or consumer touch points.
Amine Assisted Delivery (AAD): The amine-assisted delivery technology approach utilizes materials that contain an amine group to increase perfume deposition or modify perfume release during product use. There is no requirement in this approach to pre-complex or pre-react the perfume raw material(s) and amine prior to addition to the product. In one aspect, amine-containing AAD materials suitable for use herein may be non-aromatic; for example, polyalkylimine, such as polyethyleneimine (PEI), or polyvinylamine (PVAm), or aromatic, for example, anthranilates. Such materials may also be polymeric or non-polymeric. In one aspect, such materials contain at least one primary amine. This technology will allow increased longevity and controlled release also of low ODT perfume notes (e.g., aldehydes, ketones, enones) via amine functionality, and delivery of other PRMs, without being bound by theory, via polymer-assisted delivery for polymeric amines. Without technology, volatile top notes can be lost too quickly, leaving a higher ratio of middle and base notes to top notes. The use of a polymeric amine allows higher levels of top notes and other PRMS to be used to obtain freshness longevity without causing neat product odor to be more intense than desired, or allows top notes and other PRMs to be used more efficiently. In one aspect, AAD systems are effective at delivering PRMs at pH greater than about neutral. Without wishing to be bound by theory, conditions in which more of the amines of the AAD system are deprotonated may result in an increased affinity of the deprotonated amines for PRMs such as aldehydes and ketones, including unsaturated ketones and enones such as damascone. In another aspect, polymeric amines are effective at delivering PRMs at pH less than about neutral. Without wishing to be bound by theory, conditions in which more of the amines of the AAD system are protonated may result in a decreased affinity of the protonated amines for PRMs such as aldehydes and ketones, and a strong affinity of the polymer framework for a broad range of PRMs. In such an aspect, polymer-assisted delivery may be delivering more of the perfume benefit; such systems are a subspecies of AAD and may be referred to as Amine- Polymer-Assisted Delivery or APAD. In some cases when the APAD is employed in a composition that has a pH of less than seven, such APAD systems may also be considered Polymer-Assisted Delivery (PAD). In yet another aspect, AAD and PAD systems may interact with other materials, such as anionic surfactants or polymers to form coacervate and/or coacervates-like systems. In another aspect, a material that contains a heteroatom other than nitrogen, for example sulfur, phosphorus or selenium, may be used as an alternative to amine compounds. In yet another aspect, the aforementioned alternative compounds can be used in combination with amine compounds. In yet another aspect, a single molecule may comprise an amine moiety and one or more of the alternative heteroatom moieties, for example, thiols, phosphines and selenols. Suitable AAD systems as well as methods of making same may be found in US Patent Applications 2005/0003980 A1; 2003/0199422 A1; 2003/0036489 A1; 2004/0220074 A1 and U.S. Pat. No. 6,103,678.
Cyclodextrin (CD): This technology approach uses a cyclic oligosaccharide or cyclodextrin to improve the delivery of perfume. Typically a perfume and cyclodextrin (CD) complex is formed. Such complexes may be preformed, formed in-situ, or formed on or in the situs. Without wishing to be bound by theory, loss of water may serve to shift the equilibrium toward the CD-Perfume complex, especially if other adjunct ingredients (e.g., surfactant) are not present at high concentration to compete with the perfume for the cyclodextrin cavity. A bloom benefit may be achieved if water exposure or an increase in moisture content occurs at a later time point. In addition, cyclodextrin allows the perfume formulator increased flexibility in selection of PRMs. Cyclodextrin may be pre-loaded with perfume or added separately from perfume to obtain the desired perfume stability, deposition or release benefit. Suitable CDs as well as methods of making same may be found in USPA 2005/0003980 A1 and 2006/0263313 A1 and U.S. Pat. Nos. 5,552,378; 3,812,011; 4,317,881; 4,418,144 and 4,378,923.
Starch Encapsulated Accord (SEA): The use of a starch encapsulated accord (SEA) technology allows one to modify the properties of the perfume, for example, by converting a liquid perfume into a solid by adding ingredients such as starch. The benefit includes increased perfume retention during product storage, especially under non-aqueous conditions. Upon exposure to moisture, a perfume bloom may be triggered. Benefits at other moments of truth may also be achieved because the starch allows the product formulator to select PRMs or PRM concentrations that normally cannot be used without the presence of SEA. Another technology example includes the use of other organic and inorganic materials, such as silica to convert perfume from liquid to solid. Suitable SEAs as well as methods of making same may be found in USPA 2005/0003980 A1 and U.S. Pat. No. 6,458,754 B1.
Zeolite & Inorganic Carrier (ZIC): This technology relates to the use of porous zeolites or other inorganic materials to deliver perfumes. Perfume-loaded zeolite may be used with or without adjunct ingredients used for example to coat the perfume-loaded zeolite (PLZ) to change its perfume release properties during product storage or during use or from the dry situs. Suitable zeolite and inorganic carriers as well as methods of making same may be found in USPA 2005/0003980 A1 and U.S. Pat. Nos. 5,858,959; 6,245,732 B1; 6,048,830 and 4,539,135. Silica is another form of ZIC. Another example of a suitable inorganic carrier includes inorganic tubules, where the perfume or other active material is contained within the lumen of the nano- or micro-tubules. Preferably, the perfume-loaded inorganic tubule (or Perfume-Loaded Tubule or PLT) is a mineral nano- or micro-tubule, such as halloysite or mixtures of halloysite with other inorganic materials, including other clays. The PLT technology may also comprise additional ingredients on the inside and/or outside of the tubule for the purpose of improving in-product diffusion stability, deposition on the desired situs or for controlling the release rate of the loaded perfume. Monomeric and/or polymeric materials, including starch encapsulation, may be used to coat, plug, cap, or otherwise encapsulate the PLT. Suitable PLT systems as well as methods of making same may be found in U.S. Pat. No. 5,651,976.
Pro-Perfume (PP): This technology refers to perfume technologies that result from the reaction of perfume materials with other substrates or chemicals to form materials that have a covalent bond between one or more PRMs and one or more carriers. The PRM is converted into a new material called a pro-PRM (i.e., pro-perfume), which then may release the original PRM upon exposure to a trigger such as water or light. Pro-perfumes may provide enhanced perfume delivery properties such as increased perfume deposition, longevity, stability, retention, and the like. Pro-perfumes include those that are monomeric (non-polymeric) or polymeric, and may be pre-formed or may be formed in-situ under equilibrium conditions, such as those that may be present during in-product storage or on the wet or dry situs. Nonlimiting examples of pro-perfumes include Michael adducts (e.g., beta-amino ketones), aromatic or non-aromatic imines (Schiff bases), oxazolidines, beta-keto esters, and orthoesters. Another aspect includes compounds comprising one or more beta-oxy or beta-thio carbonyl moieties capable of releasing a PRM, for example, an alpha, beta-unsaturated ketone, aldehyde or carboxylic ester. The typical trigger for perfume release is exposure to water; although other triggers may include enzymes, heat, light, pH change, autoxidation, a shift of equilibrium, change in concentration or ionic strength and others. For aqueous-based products, light-triggered pro-perfumes are particularly suited. Such photo-pro-perfumes (PPPs) include but are not limited to those that release coumarin derivatives and perfumes and/or pro-perfumes upon being triggered. The released pro-perfume may release one or more PRMs by means of any of the above mentioned triggers. In one aspect, the photo-pro-perfume releases a nitrogen-based pro-perfume when exposed to a light and/or moisture trigger. In another aspect, the nitrogen-based pro-perfume, released from the photo-pro-perfume, releases one or more PRMs selected, for example, from aldehydes, ketones (including enones) and alcohols. In still another aspect, the PPP releases a dihydroxy coumarin derivative. The light-triggered pro-perfume may also be an ester that releases a coumarin derivative and a perfume alcohol. In one aspect the pro-perfume is a dimethoxybenzoin derivative as described in USPA 2006/0020459 A1. In another aspect the pro-perfume is a 3′, 5′-dimethoxybenzoin (DMB) derivative that releases an alcohol upon exposure to electromagnetic radiation. In yet another aspect, the pro-perfume releases one or more low ODT PRMs, including tertiary alcohols such as linalool, tetrahydrolinalool, or dihydromyrcenol. Suitable pro-perfumes and methods of making same can be found in U.S. Pat. Nos. 7,018,978 B2; 6,987,084 B2; 6,956,013 B2; 6,861,402 B1; 6,544,945 B1; 6,093,691; 6,277,796 B1; 6,165,953; 6,316,397 B1; 6,437,150 B1; 6,479,682 B1; 6,096,918; 6,218,355 B1; 6,133,228; 6,147,037; 7,109,153 B2; 7,071,151 B2; 6,987,084 B2; 6,610,646 B2 and 5,958,870, as well as can be found in USPA 2005/0003980 A1 and USPA 2006/0223726 A1.

Amine Reaction Product (ARP): For purposes of the present application, ARP is a subclass or species of PP. One may also use “reactive” polymeric amines in which the amine functionality is pre-reacted with one or more PRMs to form an amine reaction product (ARP). Typically the reactive amines are primary and/or secondary amines, and may be part of a polymer or a monomer (non-polymer). Such ARPs may also be mixed with additional PRMs to provide benefits of polymer-assisted delivery and/or amine-assisted delivery. Nonlimiting examples of polymeric amines include polymers based on polyalkylimines, such as polyethyleneimine (PEI), or polyvinylamine (PVAm). Nonlimiting examples of monomeric (non-polymeric) amines include hydroxylamines, such as 2-aminoethanol and its alkyl substituted derivatives, and aromatic amines such as anthranilates. The ARPs may be premixed with perfume or added separately in leave-on or rinse-off applications. In another aspect, a material that contains a heteroatom other than nitrogen, for example oxygen, sulfur, phosphorus or selenium, may be used as an alternative to amine compounds. In yet another aspect, the aforementioned alternative compounds can be used in combination with amine compounds. In yet another aspect, a single molecule may comprise an amine moiety and one or more of the alternative heteroatom moieties, for example, thiols, phosphines and selenols. The benefit may include improved delivery of perfume as well as controlled perfume release. Suitable ARPs as well as methods of making same can be found in USPA 2005/0003980 A1 and U.S. Pat. No. 6,413,920 B1.

Perfume Design (PD): The rational design of perfumes and/or perfume accords in which the physical properties of the perfume raw materials are utilized to create functional perfumes is also considered a perfume delivery technology. Such perfume design may be used with or without one or more technologies described above. When used with or without one or more of the above technologies, the technology may also be referred to as “free perfume”. Non-limiting properties that may be used in perfume design include, but are not limited to, octanol-water partition coefficient (LogP or CLopP), water solubility parameters, boiling point (bp), Kovats Index (KI) value, solvent-accessible surface area, and others. The odor detection threshold (ODT) value may also be used to design perfumes or perfume accords with preferred olfactive properties, and which may compliment or enable the use of one or more other perfume delivery technologies. Suitable perfume design (PD) systems and methods of making same can be found in US Patents 2007/0042934 A1, 2005/0003980 A1, 2007/0071780 A1, and U.S. Pat. No. 6,998,382.

While the technologies described above may be useful alone or in combination, to improve the sensory benefit of a product during the FMOT, SMOT and/or the TMOT, Applicants disclose the following combinations that may be especially useful in improving the overall sensory experience that a product provides.

| | Moment of Truth | | |
|---|---|---|---|
| Product Form | FMOT | SMOT | TMOT |
| All | Packaging and/or | Product comprising | Product comprising |
| Aspect 1 | display comprising | perfume and/or one | perfume and/or one |
| | perfume and/or one | or more perfume | or more perfume |
| | or more perfume | delivery systems | delivery systems |
| | delivery systems | | |
| All | Packaging and/or | Free perfume/ | Free perfume/ |
| Aspect 2 | display comprising | perfume design (PD), | perfume design (PD), |
| | hot melt delivery | polymer assisted | polymer assisted |
| | systems and/or | delivery (PAD), | delivery (PAD), |
| | perfume loaded | molecule-assisted | molecule-assisted |
| | plastics | delivery (MAD), | delivery (MAD), |
| | | fiber-assisted | fiber-assisted |
| | | delivery (FAB), | delivery (FAB), |
| | | amine-assisted | amine-assisted |
| | | delivery (AAD), | delivery (AAD), |
| | | cyclodextrin (CD), | zeolites & inorganic |
| | | starch encapsulated | carrier (ZIC), pro- |
| | | accord (SEA), | perfume (PP) and/or |
| | | zeolites & inorganic | amine-reaction |
| | | carrier (ZIC), and/or | product (ARP). |
| | | pro-perfume (PP). | |
| All | Packaging and/or | Free perfume/ | Free perfume |
| Aspect 3 | display comprising | perfume design, | (perfume design, |
| | hot melt delivery | perfume | perfume |
| | systems and/or | microcapsule (PMC), | microcapsule (PMC), |
| | perfume loaded | cyclodextrin, starch | nanolatex, silicone- |
| | plastics | encapsulated accord, | assisted delivery |
| | | and/or water- | (SAD), polymeric |
| | | triggered pro- | amine-assisted |
| | | perfume. | delivery, |
| | | | encapsulated/coated |
| | | | perfume loaded |
| | | | zeolites, perfume- |
| | | | loaded tubules |
| | | | (PLT), amine |
| | | | reaction product |
| | | | (ARP), and/or photo- |
| | | | pro-perfume (PPP). |
| Fluids | Packaging and/or | Product comprising | Product comprising |
| Aspect 1 | display comprising | perfume and/or one | perfume and/or one |
| | perfume and/or one | or more perfume | or more perfume |
| | or more perfume | delivery systems | delivery systems |
| | delivery systems | | |
| Aqueous Fluids | Packaging and/or | Free perfume/ | Free perfume/ |
| Aspect 1 | display comprising | perfume design, | perfume design, |
| | perfume and/or one | polymer-assisted | polymer-assisted |
| | or more perfume | delivery, amine- | delivery, monomer- |
| | delivery systems | assisted delivery | assisted delivery, |
| | | and/or pro-perfume | amine assisted |
| | | | delivery, zeolite & |
| | | | inorganic carriers, |
| | | | and/or pro-perfumes |
| Aqueous Fluids | Packaging and/or | Free perfume having | Free perfume/ |
| Aspect 2 | display comprising | PRMs with Kovats | perfume design, |
| | hot melt delivery | Index values of less | microcapsules, |
| | systems and/or | than about 1500, | nanolatex, silicone |
| | perfume loaded | from less than about | assisted delivery, |
| | plastics | 1500 to about 800, or | polymeric amine |
| | | even from less than | assisted delivery, |
| | | about 1400 to about | perfume-loaded |
| | | 1000 and/or | zeolites, perfume |
| | | microcapsules. | loaded tubules, photo |
| | | | and/or enzyme |
| | | | triggered pro- |
| | | | perfumes |
| Non-Aqueous | Packaging and/or | Free perfume/ | Free perfume/ |
| Aspect 1 | display comprising | perfume design, | perfume design, |
| | perfume and/or one | cyclodextrin, starch | polymer assisted |
| | or more perfume | encapsulated accords, | delivery, monomer- |
| | delivery systems | polymer-assisted | assisted delivery, |
| | | delivery and/or pro- | amine assisted |
| | | perfumes | delivery, zeolite & |
| | | | inorganic carriers, |
| | | | and/or pro-perfumes |
| Non-Aqueous | Packaging and/or | Free perfume having | Free perfume/ |
| Aspect 2 | display comprising | PRMs with Kovats | perfume design, |
| | perfume and/or one | Index values of less | microcapsules, |
| | or more perfume | than about 1500, | perfume-loaded |
| | delivery systems | from less than about | zeolites, perfume |
| | | 1500 to about 800, or | loaded tubules, |
| | | even from less than | amine-reaction |
| | | about 1400 to about | products, and/or |
| | | 1000, cyclodextrin, | photo-pro-perfumes |
| | | starch encapsulated | |
| | | accords and/or water- | |
| | | triggered pro- | |
| | | perfumes | |
| Solids | Packaging and/or | Product comprising | Product comprising |
| Aspect 1 | display comprising | perfume and/or one | perfume and/or one |
| | perfume and/or one | or more perfume | or more perfume |
| | or more perfume | delivery systems | delivery systems |
| | delivery systems | | |
| Solids | Packaging and/or | Free perfume/ | Free perfume/ |
| Aspect 2 | display comprising | perfume design, | perfume design, |
| | hot melt delivery | cyclodextrin, starch | polymer assisted |
| | systems and/or | encapsulated accords, | delivery, monomer- |
| | perfume loaded | polymer-assisted | assisted delivery, |
| | plastics | delivery and/or pro- | amine assisted |
| | | perfumes | delivery, zeolite & |
| | | | inorganic carriers, |
| | | | and/or pro-perfumes |
| Solids | Packaging and/or | Free perfume having | Free perfume/ |
| Aspect 3 | display comprising | PRMs with Kovats | perfume design, |
| | hot melt delivery | Index values of less | microcapsules, |
| | systems and/or | than about 1500, | perfume-loaded |
| | perfume loaded | from less than about | zeolites, perfume |
| | plastics | 1500 to about 800, or | loaded tubules, |
| | | even from less than | amine-reaction |
| | | about 1400 to about | products, and/or |
| | | 1000, cyclodextrin, | photo-pro-perfumes |
| | | starch encapsulated | |
| | | accords and/or water- | |
| | | triggered pro- | |
| | | perfumes | |
| Apparatus | Packaging and/or | Product comprising | Product comprising |
| Aspect 1 | display comprising | perfume and/or one | perfume and/or one |
| | perfume and/or one | or more perfume | or more perfume |
| | or more perfume | delivery systems | delivery systems |
| | delivery systems | | |
| Apparatus | Packaging and/or | Free perfume/ | Free perfume/ |
| Aspect 2 | display comprising | perfume design, | perfume design, |
| | perfume and/or one | cyclodextrin, starch | polymer assisted |
| | or more perfume | encapsulated accords, | delivery, monomer- |
| | delivery systems | polymer-assisted | assisted delivery, |
| | | delivery and/or pro- | amine assisted |
| | | perfumes | delivery, zeolite & |
| | | | inorganic carriers, |
| | | | and/or pro-perfumes |
| Apparatus | Packaging and/or | Free perfume/ | Free perfume/ |
| Aspect 3 | display comprising | perfume design, | perfume design, |
| | hot melt delivery | cyclodextrin, and/or | microcapsules, |
| | systems and/or | starch encapsulated | perfume-loaded |
| | perfume loaded | accords | zeolites, perfume |
| | plastics | | loaded tubules, |
| | | | amine-reaction |
| | | | products, and/or |
| | | | photo-pro-perfumes |

Additional aspects are identical to the aspects disclosed in Table 1 above except such additional aspects do not include the FMOT technology, for example, such aspects do not include packaging and/or a display comprising perfume and/or one or more perfume delivery systems.

Thus, aspects of a packaged product may comprise the following:

### All Aspect 1

In one aspect, a packaged product is disclosed wherein the packaging may comprise a perfume packaging delivery system selected from the group consisting of free perfume, one or more perfume delivery systems and mixtures thereof; and the product matrix may comprise a free perfume and/or one or more perfume delivery systems. In such aspect, the packaging may comprise, based on total packaging weight, from about 0.001% to about 10%, from about 0.03% to about 3%, or even from about 0.03% to about 0.3% free perfume and/or from about 0.001% to about 50%, from about 0.01% to about 20%, or even from about 0.01% to about 5% of one or more perfume packaging delivery systems and the product matrix may comprise, based on total product matrix weight, from about 0.001% to about 30%, from about 0.05% to about 10%, or even from about 0.1% to about 3% of a free perfume and/or from about 0.001% to about 60%, from about 0.05% to about 25%, or even from about 0.1% to about 8% of one or more perfume delivery systems. In the aforementioned aspect, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system for the product matrix may be from about 1:1000 to about 20:1, from about 1:20 to about 10:1 or even from about 1:10 to about 5:1.

### All Aspect 2

In another aspect, a packaged product is disclosed wherein the packaging may comprise a perfume packaging delivery system selected from the group consisting of a hot melt delivery system, a perfume loaded plastic and mixtures thereof; and the product matrix may comprise a perfuming material selected from the group consisting of free perfume, polymer assisted delivery (PAD), molecule-assisted delivery (MAD), amine-assisted delivery (AAD), fiber-assisted delivery (FAD), cyclodextrin (CD), starch encapsulated accord (SEA), zeolite/inorganic carrier system (ZIC), and pro-perfume (PP) system, and mixtures thereof. In such aspect, the packaging may comprise, based on total packaging weight, from about 0.001% to about 50%, from about 0.01% to about 20%, or even from about 0.01% to about 5% of said perfume packaging delivery system and said product matrix may comprise, based on total product matrix weight, from about 0.001% to about 60%, from about 0.05% to about 25%, or even from about 0.1% to about 8% of said perfuming material. In the aforementioned aspect, when the packaging comprises free perfume, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system for the product matrix may be from about 1:1000 to about 20:1, from about 1:20 to about 10:1 or even from about 1:10 to about 5:1.

### All Aspect 3

In another aspect, a packaged product is disclosed wherein the packaging may comprise a perfume packaging delivery system selected from the group consisting of a hot melt delivery system, a perfume loaded plastic and mixtures thereof; and the product matrix may comprise a perfuming material selected from the group consisting of free perfume, perfume microcapsule (PMC), cyclodextrin, starch encapsulated accord, water-triggered pro-perfume, nanolatex, silicone-assisted delivery (SAD), polymeric amine-assisted delivery, encapsulated or coated perfume loaded zeolites, perfume-loaded tubules (PLT), amine reaction product (ARP), photo-pro-perfume (PPP) and mixtures thereof. In such aspect, the packaging may comprise based on total packaging weight, from about 0.001% to about 50%, from about 0.01% to about 20%, or even from about 0.01% to about 5% of said perfume packaging delivery system and said product matrix may comprise, based on total product matrix weight, from about 0.001% to about 60%, from about 0.05% to about 25%, or even from about 0.1% to about 8% of said perfuming material. In the aforementioned aspect, when the packaging comprises free perfume, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system in the product matrix may be from about 1:1000 to about 20:1, from about 1:20 to about 10:1 or even from about 1:10 to about 5:1.

### Fluid Aspect 1

In another aspect, a packaged product is disclosed wherein the packaging may comprise a perfume packaging/display delivery system selected from the group consisting of free perfume, one or more perfume delivery systems and mixtures thereof; and the product matrix may comprise a fluid and said fluid may comprise a free perfume and/or one or more perfume delivery systems. In such aspect, the packaging may comprise based on total packaging weight, from about 0.001% to about 10%, from about 0.03% to about 3%, or even from about 0.03% to about 0.3% free perfume and/or from about 0.001% to about 50%, from about 0.01% to about 20%, or even from about 0.01% to about 5% of one or more perfume packaging delivery systems and the product matrix may comprise, based on total product matrix weight, from about 0.001% to about 30%, from about 0.05% to about 10%, or even from about 0.1% to about 3% of a free perfume and/or from about 0.001% to about 30%, from about 0.05% to about 15%, or even from about 0.1% to about 5% of one or more perfume delivery systems. In the aforementioned aspect, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system for the product matrix may be from about 1:1000 to about 20:1, from about 1:20 to about 10:1 or even from about 1:10 to about 5:1.

### Aqueous Fluid Aspect 1

In another aspect, a packaged product is disclosed wherein the packaging may comprise a perfume packaging delivery system selected from the group consisting of free perfume and/or one or more perfume delivery systems and the product matrix, based on total product matrix weight, may comprise from about 2% to about 99.9%, from about 5% to about 99%, from about 10% to about 98%, from about 20% to about 95%, or even from about 25% to about 90% water and a perfuming material selected from the group consisting of free perfume, polymer-assisted delivery, amine-assisted delivery, pro-perfume, molecule-assisted delivery, zeolite/inorganic carrier system, and mixtures thereof. In such aspect, the packaging may comprise based on total packaging weight, from about 0.001% to about 10%, from about 0.03% to about 3%, or even from about 0.03% to about 0.3% free perfume and/or from about 0.001% to about 50%, from about 0.01% to about 20%, or even from about 0.01% to about 5% of one or more perfume packaging delivery systems and said product matrix may comprise, based on total product matrix weight, from about 0.001% to about 60%, from about 0.05% to about 25%, or even from about 0.1% to about 8% of said perfuming material. In the aforementioned aspect, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system in the product matrix may be from about 1:1000 to about 20:1, from about 1:20 to about 10:1 or even from about 1:10 to about 5:1.

### Aqueous Fluid Aspect 2

In another aspect, a packaged product is disclosed wherein the packaging may comprise a perfume packaging delivery system selected from the group consisting of a hot melt delivery system, a perfume loaded plastic and mixtures thereof; and the product matrix may comprise, based on total product matrix weight, from about 2% to about 99.9%, from about 5% to about 99%, from about 10% to about 98%, from about 20% to about 95%, or even from about 25% to about 90%, water and a perfuming material selected from the group consisting of a free perfume having PRMs with Kovats Index values of less than about 1500, from less than about 1500 to about 800, or even from less than about 1400 to about 1000, cyclodextrin, starch encapsulated accords, water-triggered pro-perfumes, microcapsules, perfume-loaded zeolites, perfume loaded tubules, amine-reaction products, photo-pro-perfumes delivery system and mixtures thereof. In such aspect, the packaging may comprise based on total packaging weight, from about 0.001% to about 50%, from about 0.01% to about 20%, or even from about 0.01% to about 5% of said perfume packaging delivery system and said product matrix may comprise, based on total product matrix weight, from about 0.001% to about 60%, from about 0.05% to about 25%, or even from about 0.1% to about 8% of said perfuming material. In the aforementioned aspect, the product matrix, may comprise, based on total product matrix weight, 0.0001% to about 60%, from about 0.005% to about 25%, or even from about 0.01% to about 8% of said perfume having a Kovats Index of less than about 1500, from less than about 1500 to about 800, or even from less than about 1400 to about 1000. In the aforementioned aspect, when the packaging comprises free perfume, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system in the product matrix may be from about 1:1000 to about 20:1, from about 1:20 to about 10:1 or even from about 1:10 to about 5:1.

### Non-Aqueous Fluid Aspect 1

In another aspect, a packaged product is disclosed wherein the packaging may comprise a perfume packaging delivery system selected from the group consisting of free perfume and/or one or more perfume delivery systems and the product matrix comprises a may comprise a non-aqueous fluid and a perfuming material selected from the group consisting of free perfume, cyclodextrin, starch encapsulated accords, polymer-assisted delivery, pro-perfumes, polymer assisted delivery, molecule-assisted delivery, amine assisted delivery, zeolite/inorganic carrier delivery system and mixtures thereof. In such aspect, the packaging may comprise, based on total packaging weight, from about 0.001% to about 10%, from about 0.03% to about 3%, or even from about 0.03% to about 0.3% free perfume and/or from about 0.001% to about 50%, from about 0.01% to about 20%, or even from about 0.01% to about 5% of one or more perfume packaging delivery systems and said product matrix may comprise, based on total product matrix weight, from about 0.001% to about 60%, from about 0.05% to about 25%, or even from about 0.1% to about 8% of said perfuming material. In the aforementioned aspect, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system in the product matrix may be from about 1:1000 to about 20:1, from about 1:20 to about 10:1 or even from about 1:10 to about 5:1.

### Non-aqueous Fluid Aspect 2

In another aspect, a packaged product is disclosed wherein the packaging may comprise a perfume packaging delivery system selected from the group consisting of a hot melt delivery systems a perfume loaded plastic and mixtures thereof; and the product matrix may comprise based on total product matrix weight, from about 2% to about 99.9%, from about 5% to about 99%, from about 10% to about 98%, from about 20% to about 95%, or even from about 25% to about 90%, of a non-aqueous fluid and a perfuming material selected from the group consisting of a free perfume having PRMs with Kovats Index values of less than about 1500, from less than about 1500 to about 800, or even from less than about 1400 to about 1000, cyclodextrin, starch encapsulated accords, water-triggered pro-perfumes, microcapsules, perfume-loaded zeolites, perfume loaded tubules, amine-reaction products, photo-pro-perfumes delivery system, and mixtures thereof. In such aspect, the packaging may comprise, based on total packaging weight, from about 0.001% to about 50%, from about 0.01% to about 20%, or even from about 0.01% to about 5% of said perfume packaging delivery system and said product matrix may comprise, based on total product matrix weight, from about 0.001% to about 60%, from about 0.05% to about 25%, or even from about 0.1% to about 8% of said perfuming material. In the aforementioned aspect, the product matrix may comprise, based on total product matrix weight, 0.0001% to about 60%, from about 0.005% to about 25%, or even from about 0.01% to about 8% of said perfume having a Kovats Index of less than about 1500, from less than about 1500 to about 800, or even from less than about 1400 to about 1000. In the aforementioned aspect, when the packaging comprises free perfume, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system in the product matrix may be from about 1:1000 to about 20:1, from about 1:20 to about 10:1 or even from about 1:10 to about 5:1.

### Solids Aspect 1

In another aspect, a packaged product is disclosed wherein the packaging may comprise a perfume packaging delivery system selected from the group consisting of free perfume, one or more perfume delivery systems and mixtures thereof; and the product matrix may comprise a solid, and a free perfume and/or one or more perfume delivery systems. All or a portion of said free perfume and/or one or more perfume delivery systems may be found in said solid. In such aspect, the packaging may comprise, based on total packaging weight, from about 0.001% to about 10%, from about 0.03% to about 3%, or even from about 0.03% to about 0.3% free perfume and/or from about 0.001% to about 50%, from about 0.01% to about 20%, or even from about 0.01% to about 5% of one or more perfume packaging delivery systems and the product matrix may comprise, based on total product matrix weight, from about 0.001% to about 30%, from about 0.05% to about 10%, or even from about 0.1% to about 3% of a free perfume and/or from about 0.001% to about 30%, from about 0.05% to about 15%, or even from about 0.1% to about 5% of one or more perfume delivery systems. In the aforementioned aspect, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system for the product matrix may be from about 1:1000 to about 20:1, from about 1:20 to about 10:1 or even from about 1:10 to about 5:1.

### Solids Aspect 2

In another aspect, a packaged product is disclosed wherein the packaging may comprise a perfume packaging delivery system selected from the group consisting of a hot melt delivery system, a perfume loaded plastic and mixtures thereof; and the product matrix may comprise a solid, and a perfuming material selected from the group consisting of free perfume, cyclodextrin, starch encapsulated accords, polymer-assisted delivery, pro-perfumes, molecule-assisted delivery, amine assisted delivery, zeolite/inorganic carrier delivery system and mixtures thereof. All or a portion of said perfuming material may be found in said solid. In such aspect, the packaging may comprise, based on total packaging weight, from about 0.001% to about 50%, from about 0.01% to about 20%, or even from about 0.01% to about 5% of said perfume packaging delivery system and said product matrix may comprise, based on total product matrix weight, from about 0.001% to about 60%, from about 0.05% to about 25%, or even from about 0.1% to about 8% of said perfuming material. In the aforementioned aspect, when the packaging comprises free perfume, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system for the product matrix may be from about 1:1000 to about 20:1, from about 1:20 to about 10:1 or even from about 1:10 to about 5:1.

### Solid Aspect 3

In another aspect, a packaged product is disclosed wherein the packaging may comprise a perfume packaging delivery system selected from the group consisting of a hot melt delivery system, a perfume loaded plastic and mixtures thereof; and the product matrix may comprise a solid and perfuming material selected from the group consisting of a free perfume having PRMs with Kovats Index values of less than about 1500, from less than about 1500 to about 800, or even from less than about 1400 to about 1000, cyclodextrin, starch encapsulated accords, water-triggered pro-perfumes, microcapsules, perfume-loaded zeolites, perfume loaded tubules, amine-reaction products, photo-pro-perfumes, and mixtures thereof. All or a portion of said perfuming material may be found in said solid. In such aspect, the packaging may comprise, based on total packaging weight, from about 0.001% to about 50%, from about 0.01% to about 20%, or even from about 0.01% to about 5% of said perfume packaging delivery system and said product matrix may comprise, based on total product matrix weight, from about 0.001% to about 60%, from about 0.05% to about 25%, or even from about 0.1% to about 8% of said perfuming material. In the aforementioned aspect, the product matrix may comprise, based on total product matrix weight, 0.0001% to about 60%, from about 0.005% to about 25%, or even from about 0.01% to about 8% of said perfume having a Kovats Index of less than about 1500, from less than about 1500 to about 800, or even from less than about 1400 to about 1000. In the aforementioned aspect, when the packaging comprises free perfume, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system in the product matrix may be from about 1:1000 to about 20:1, from about 1:20 to about 10:1 or even from about 1:10 to about 5:1.

### Apparatus Aspect 1

In another aspect, a packaged apparatus is disclosed wherein the packaging may comprise a perfume packaging delivery system selected from the group consisting of free perfume, one or more perfume delivery systems and mixtures thereof; and the apparatus may comprise a free perfume and/or one or more perfume delivery systems. In such aspect, the packaging may comprise, based on total packaging weight, from about 0.001% to about 10%, from about 0.03% to about 3%, or even from about 0.03% to about 0.3% free perfume and/or from about 0.001% to about 50%, from about 0.01% to about 20%, or even from about 0.01% to about 5% of one or more perfume packaging delivery systems and the apparatus may comprise, based on total apparatus weight, from about 0.001% to about 30%, from about 0.05% to about 10%, or even from about 0.1% to about 3% of a free perfume and/or from about 0.001% to about 30%, from about 0.05% to about 15%, or even from about 0.1% to about 5% of one or more perfume delivery systems. In the aforementioned aspect, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system for the apparatus may be from about 1:1000 to about 20:1, from about 1:20 to about 10:1 or even from about 1:10 to about 5:1.

### Apparatus Aspect 2

In another aspect, a packaged apparatus is disclosed wherein the packaging may comprise a perfume packaging delivery system selected from the group consisting of free perfume, cyclodextrin, starch encapsulated accords, polymer-assisted delivery, pro-perfumes, molecule-assisted delivery, amine assisted delivery, zeolite/inorganic carrier delivery systems and mixtures thereof. In such aspect, the packaging may comprise, based on total packaging weight, from about 0.001% to about 50%, from about 0.01% to about 20%, or even from about 0.01% to about 5% of said perfume packaging delivery system and said apparatus may comprise, based on total apparatus weight, from about 0.001% to about 60%, from about 0.05% to about 25%, or even from about 0.1% to about 8% of said perfuming material. In the aforementioned aspect, when the packaging comprises free perfume, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system for the apparatus may be from about 1:1000 to about 20:1, from about 1:20 to about 10:1 or even from about 1:10 to about 5:1.

### Apparatus Aspect 3

In another aspect, a packaged apparatus is disclosed wherein the packaging may comprise a perfume packaging delivery system selected from the group consisting of a hot melt delivery system, a perfume loaded plastic and mixtures thereof; and the apparatus may comprise a perfuming material selected from the group consisting of free perfume, cyclodextrin, starch encapsulated accords, microcapsules, perfume-loaded zeolites, perfume loaded tubules, amine-reaction products, photo-pro-perfumes and mixtures thereof. In such aspect, the packaging may comprise, based on total packaging weight, from about 0.001% to about 50%, from about 0.01% to about 20%, or even from about 0.01% to about 5% of said perfume packaging delivery system and said apparatus may comprise, based on total apparatus weight, from about 0.001% to about 60%, from about 0.05% to about 25%, or even from about 0.1% to about 8% of said perfuming material. In the aforementioned aspect, when the packaging comprises free perfume, the ratio of free perfume to perfume delivery system for the package may be from about 1:100 to about 20:1, from about 1:20 to about 10:1 or even from about 1:10 to about 5:1. In the aforementioned aspect, the ratio of free perfume to perfume delivery system in the product matrix may be from about 1:1000 to about 20:1, from about 1:20 to about 10:1 or even from about 1:10 to about 5:1.

In one or more aspects the packaged product of Aspect 1 above may comprise a PAD reservoir system in the form of a perfume microcapsule and a PAD matrix system and/or an amine assisted delivery (AAD) system.

In one or more aspects, the perfume delivery system may comprise one or more substrates wherein the perfume delivery technology is applied to said one or more substrates or wherein the perfume delivery technology is sandwiched between said one or more substrates. In one aspect, said one or more substrates comprise one or more perfume delivery technologies such as a polymeric matrix system. In one aspect, said sandwiched perfume delivery technology may be a microcapsule, such as perfume microcapsule.

In one or more aspects the packaged product of Aspect 1 above may comprise a pro-perfume, for example, a photo pro-perfume (PPP) and a PAD reservoir system, a PAD matrix system and/or an amine assisted delivery (AAD) system. In another aspect, the PAD reservoir system is a perfume microcapsule (PMC); and even a perfume microcapsule wherein the capsule wall is based on a urea-formaldehyde and/or a melamine-formaldehyde resin. In still another aspect, the AAD system comprises a material selected from the group of polyethyleneimine (PEI) and polyvinylamine (PVAm).

In one or more aspects the packaged product of Aspect 1 above may comprise nanotubules. Such packaged product may also comprise a material selected from the group consisting of a polymer assisted delivery system, an amine assisted delivery system, an amine reaction product, a cyclodextrin, a starch encapsulated accord, a perfume loaded zeolite, a coated perfume loaded zeolite, a pro-perfume and mixtures thereof.

In additional aspects, such additional aspects are identical to the aspects disclosed above except the perfume packaging delivery system that is associated with any packaging is optional. In short, such aspects may be free of a perfume packaging delivery system.

In other additional aspects, such additional aspects are identical to the aspects disclosed above except the packaging is optional. In short, such aspects may be free of packaging.

In additional aspects, such aspects are identical to the aspects disclosed above and such aspects are associated with a display that may comprise a perfume display delivery system that is identical to the perfume packaging delivery system disclosed with said aspects disclosed above.

In the aforementioned aspects, multiple FMOT technologies may be employed as the experience provided by a single FMOT technology may, at times, be insufficient to meet all the needs of the consumer. For example, the perfume release provided by a single packaging approach or FMOT perfume delivery technology (PDT) may not be experienced or noticed by every consumer. For example, one consumer may lift a product's cap and experience the benefit of a FMOT PDT, such as a matrix-based polymer-assisted delivery (PAD) technology (for example, a perfume hot melt or a perfumed plastic insert); however another consumer may not decide to lift the cap, and would instead be more likely to notice a FMOT PDT that is positioned on the outside of the product or package. Such a FMOT PDT could be selected from the list comprising 1) PAD matrix type hot melts, 2) perfume injected plastic, 3) perfumed labels, 4) scratch & sniff perfume release systems, or even 5) perfume samplers that are present in the proximity of the product, such as part of a store display. Any of these FMOT PDTs could be independently selected, and be independently positioned on the product, such as on the top, side or bottom of the product and/or package.

Another example of a solution to this need for improved FMOT experience is using a same or similar PDT, such as perfume hot melts, but such that the same or similar PDT is present in more than one location on the product. For example, the hot melt could be present under the lid of the product and the same or different FMOT PDT could be on the bottom of the product. Based on the desired experience, the perfume selected for incorporation into one FMOT PDT (e.g., hot melt or perfumed plastic insert) may be the same, may be similar or may be different in perfume composition as the perfume selected for incorporation into one or more other hot melts contained as part of the product and/or product packaging.

Hot melt or adhesive-based systems will release perfume raw materials (PRMs) at a different rate than alternative FMOT PDTs such as perfume injected plastics or the like. The difference in perfume release profiles can be leveraged to provide greater olfactive benefit than for a single PDT in which certain compromises would need to be struck by the perfumer or product formulator in order to balance factors such as character, intensity, release profile, cost, and the like. For example, one combination option is to include the same FMOT PDT on different parts of the product but select different perfume accords for different samples. For example, one FMOT PDT could contain a higher percentage of top notes with a Kovats index (KI) value in the range of 800 to 1200, whereas another hot melt might contain more middle notes or PRMs with a Kovats index (KI) value in the range of 1200-1500, whereas yet another might contain base notes with a Kovats index (KI) value>1500. This would have the advantage of allowing one to select the perfume loading based on release profile of the PRMs, which have different volatilities.

For any of the FMOT PDTs, such as hot melts or perfume plastics, the system can be improved by selecting key PRMs for release that are based on combination of parameters such as Kovats Index (KI) and Odor Detection Threshold (ODT). Preferred range of KI for PRMS may be from 800 to 1500, more preferably from 1000 to 1400, even more preferably from 1000 to 1200. In addition, one may choose individual PRMs or perfume accords to enhance the scent of the perfume in the product. These PRMs may be selected from those PRMs that may not be easy to formulate due to chemical instability, for example. Other examples of perfume design in which different PRMs or accords are selected for different FMOT PDTs and/or the perfume in the product also fall within the scope of this invention.

Another aspect of the present invention includes the use of combinations of FMOT PDTs to provide a desired experience at the point of purchase for products that contain low perfume levels, or no perfume. Such products may be chosen by the consumer to minimize exposure to perfume during in-use experience of for the dry situs. Such products with low in-product perfume levels may have an undesired odor due to the rest of the formulation ingredients. As such, the FMOT PDT or PDTs may be used to improve the scent experience at the point of purchase.

In the aforementioned aspects, it is also important for the consumer to have an ideal scent experience when using the product. For example, the consumer should be pleased by the fresh character and preferred intensity of the scent of the product during use. While FMOT PDT or PDTs typically serve the need of providing the desired sensory experience at point of purchase, they may not deliver throughout the in-use experience, for example, because the product may be put away during use, or the treated situs is put away before use, or the scent intensity may be insufficient in strength to meet the consumer need during use. In addition, the multiple consumer touch points during the in-use experience may require additional PDTs to drive the desired sensory experience. For example, one SMOT PDT may be needed to provide desired product scent experience when opening package and/or when pouring the product out of box or bottle, and another SMOT PDT to maintain the desired scent intensity and character of the wash solution. In addition, the same or different SMOT PDT(s) may be selected to provide scent that fills and lingers in the room in which the product is being used, as well as, to provide scent to the treated situs. For example, in the case of a laundry detergent or fabric conditioners, the SMOT PDT can provide a desired scent experience on the wet laundry, particularly when it is taken out of the washer. For consumers that wash fabrics by hand, such in-use perfume release (a.k.a., perfume bloom) is particularly important to the scent experience. Other examples include hair and skin care products, including but not limited to, hair shampoos and conditioners, body washes, including those with one or more separate phases, bar soaps, antiperspirants, deodorants, and the like. For such products, the perfume bloom during the use of the product in its neat or water-diluted form is important to the SMOT consumer experience.

Using SMOT PDT or PDTs may provide increased perfume intensity or perfume bloom during product use, including in cases in which the perfume release is triggered by moisture, such that the scent may be experienced, even filling the room, when the product package is not present or when the FMOT PDT is not providing sufficient scent in combination with the perfume associated with the neat product.

Any of the PDTs described within may be used in any combination in a consumer product to achieve the desired FMOT or SMOT scent experience. Examples of SMOT PDTs include starch encapsulated accords, cyclodextrin, pro-perfumes, perfume-loaded polymers, which include matrix (e.g., nanolatex) and reservoir (microcapsule) systems.

SMOT PDTs in which perfume release is triggered by an increase in moisture during product use are particularly suited for low moisture product forms for example granular detergents, fabric softener sheets, laundry tablets, and the like.

In many cases, SMOT PDTs may decrease the neat product odor (NPO) or perfume headspace above the product, without being bound by theory, due to the interaction between the carrier and the perfume raw materials (PRMs). One skilled in the art can attempt to compensate for the loss of perfume headspace by adding additional free perfume; however, this approach can add to the expense of the product and can negatively impact the consumer experience at other consumer touch points by causing the scent to be distorted either in terms of character or intensity. The use of the present invention, which combines FMOT and SMOT PDTs, may address the problem caused by the use of said SMOT or TMOT PDT that is intended to provide benefit during or after product use. For example, cyclodextrin and/or SEA may be individually loaded with perfume and formulated into a granular laundry detergent, which may provide a burst or bloom of perfume intensity upon product use. The scent intensity and character of the neat product, however, may be insufficient or distorted in such a way as to decrease consumer acceptance at the FMOT (point of purchase). As such, addition of a FMOT PDT solves the problem by allowing the formulator to improve the overall scent experience. This may include a small amount of perfume present on the product or package that is closer in scent character to the scent of the wash solution once some or all of the perfume is released from the SMOT PDT. Alternatively, a perfume with a different character and intensity may be selected for incorporation into the FMOT PDT.

In another aspect, a means to overcome the challenge of the consumer becoming tired of the same scent being detected from the neat product odor, to the in-use experience, and even to the after use experience is disclosed. This invention allows one to provide different perfume characters at each consumer touch point. Thus, the perfume of the packaging material may be different than the perfume in the bottle and optionally different from the perfume released during product use and optionally different from the perfume that is released from the wet situs or from the delivery system releasing perfume from the wet or dry situs after product use. Thus, it enables the formulator to balance the overall scent experience of the consumer by combining any or all of the following PDT(s) described herein.

Another advantage of the present invention is that the release profile or release rate may be modified by using combinations of FMOT and SMOT PDTs. A particular PRM may have properties that make it difficult to provide a desired headspace level, for example, if the PRM has a low vapor pressure when present with other formulation ingredients such as surfactant. The use of a FMOT PDT may allow such PRMs to be present in the headspace near the product such that the benefit of said PRM may be realized.

Employing multiple SMOT PDTS may be useful. Although certain perfume carriers are effective at complexing a broad range of PRMs, many are limited by which PRMs are fully complexed. Such limitations may be associated with molecular size or shape, for example. In such cases, it is necessary to compliment a given SMOT PDT with additional SMOT PDTs. For example, for certain highly volatile PRMs, there may be loses during the starch encapsulation process. Such PRMs however may be more readily incorporated in cyclodextrin, especially if they have a high binding constant. As such, the combination of SEA and CD in a product may provide greater scent benefits in terms of character and intensity than either could provide separately.

Although products utilizing FMOT PDT(s) may improve consumer experience at point of purchase, and SMOT PDTs may provide an improved in-use experience, there is also a need to improve scent of the dry situs. For example, there is an unmet need to provide improved dry fabric odor (DFO), both in terms of scent character and intensity. Thus, TMOT PDTs are useful. For many TMOT PDTs there is a strong interaction between the carrier and the perfume. In order to achieve improved situs odor, it is necessary to increase deposition of PRMs onto the situs. This may be achieved by using a carrier technology, however this carrier may suppress neat product odor, such that the FMOT experience is distorted. This problem is solved by the current invention in which the overall experience is enhanced by using FMOT PDT(s) in combination with TMOT PDT(s).

For example, the encapsulation of perfume by a polymer wall or matrix allows the perfume to be protected from harsh product conditions and allows increased perfume deposition to the situs. Without being bound by theory, the encapsulation technology can suppress the perfume headspace prior to the release of the perfume from the carrier due to a trigger. Such triggers include but are not limited to diffusion, friction, heat, dilution, ionic strength, water, pH, light, and the like. The suppression of perfume headspace can lead to a distortion of the neat product odor and the FMOT consumer experience. The use of a FMOT PDT solves this problem by improving the character and intensity at the earlier consumer touch point or moment of truth, and using the TMOT PDT(s) to provide benefits to the treated situs, for example DFO.

Similar to perfume microcapsules (PMCs), the use of FMOT PDT may provide similar advantage when using other TMOT PDTs described in this application. These include, but are not limited to, pro-perfumes, perfume-loaded zeolites (PLZ), other polymer-assisted delivery systems, such as nanolatex, amine-assisted delivery technologies, and nanotubules.

Employing multiple TMOT PDTs may be useful. There are many consumer touch points associated with TMOT that often will require the use of more than one PDT to satisfy the consumer need. For example, for fabric applications, different TMOT PDTs may be required to improve character and intensity of dry or nearly dry laundry as it is 1) removed from the dryer, 2) taken off the drying line, 3) folded and stored, 4) ironed, 5) freshly put on, 6) smelled by the consumer or others after hours of wearing, 7) re-worn after having been worn or used previously, and 8) removed from the dirty laundry pile prior to laundering. Another example includes hair or skin (or scalp) applications, wherein different TMOT PDTs may be required to improve character and intensity of dry or nearly dry hair or skin as the consumer is noticing the scent of the perfume on themselves or others 1) during or after drying off, for example, with a towel and/or hair or hand dryer following the use of the product for washing, bathing, showering or the like, 2) after exiting the room or area in which the product was used (e.g., bathroom or shower), 3) after returning to the location the product was used, 4) after hours since the use of the product, or 5) after one or more days since the use of the product. Other consumer touch points associated with TMOT include, but are not limited to, the scent on fabric after using hair or skin products, including antiperspirants and deodorants, and the change in scent intensity and/or character that may be associated with changes in the moisture content of a situs. All of the above consumer touch points can be addressed using combinations of perfume delivery technologies that provide a benefit after a single use or application or after multiple uses or applications.

It is not uncommon for certain PRMs to “leak” out of perfume microcapsules (PMC) during product storage or during product use. This invention describes a means to use a PDT to interact with or “scavenge” PRMs that may be delivered with less efficiency as desired by the PMC, to provide a superior perfume experience compared to using a PMC or a PAD individually. For some PMC systems, PRMs that leak most readily include those with low CLogP value and/or low Kovats Index (KI) value, including PRMs that are referred to as “top notes”; PAD and AAD PDTs may be used to improve the delivery of such PRMs. Another option to leverage TMOT combinations is to add additional free PRMs, including top notes, separately from the PMC, and use a PAD and/or AAD technology to improve the deposition of said separately added PRMs, in combination with a PMC system to improve the deposition and/or release of other PRMs. The PAD and/or AAD system that may be used to improve also the deposition of the PMC may be pre-loaded with perfume or added separately from the perfume to the product matrix, thus improving both deposition of free PRMs and PMC, and optionally modifying the release profile of free perfume and/or encapsulated perfume from the situs.

In addition to balancing the overall character and intensity by using combinations of TMOT PDTs to delivery different perfumes, perfume accords, or PRMs, another advantage to using combinations of TMOT PDTs is to improve the overall deposition and release profile of PDTs. For example, cationic polymers and/or other additives may be used to increase the deposition of PMC on the situs. In addition such additives can modify the release profile of perfume from PMC. Without being bound by theory, the perfume release may be modified by the ability of the additive to increase or decrease the wall porosity or modify the diffusivity of the wall to the internal or external phase. Also, without being bound by theory, the additive may serve as a perfume sink for PRMs that are released from the PMC either prior to or after a triggered release.

In another aspect of the present invention, select PRMs are able to diffuse through the wall of the PMC. Such diffusion may be desirable to achieve sustained perfume release; however, in other cases the release of such PRMs is considered too fast and results in a deficiency of certain PRMs and a change in the preferred character or intensity. The use of an additional TMOT PDT, such as a pro-perfume, allows the desired character to be maintained over time by providing sustained perfume release, including of those PRMs that are able to diffuse through the wall of the PMC. Thus, PRMs that are not effectively delivered by PMC or need to be delivered at a different or modified headspace concentration may be delivered by means of a TMOT PDT based on pro-perfume technology.

Another aspect of the present invention uses TMOT PDT to enhance the deposition of one or more other TMOT PDTs. For example, polymers, preferably cationic polymers may be formulated into the product in order to increase the deposition of specific PRMs and also increase the deposition of another TMOT PDT, such as a pro-perfume. Such pro-perfumes include light-triggered pro-perfumes. The polymer not only serves to increase deposition, but also serves as a perfume carrier to deliver additional PRMs to the situs. The polymer may also serve to modify the release profile of the PRM or PRMs released from the pro-perfume.

Other examples of improved deposition may be achieved by combining PMC, pro-perfumes, zeolite or clays, polymer-assisted delivery (e.g., a nanolatex), amine-assisted delivery, inorganic nanotubules, photo-pro-perfumes, silicones, and the like.

Another aspect of the present invention allows the formulator to combine TMOT PDTs to maximize freshness or minimize scent polarization. Pro-perfume for example may deliver a single PRM or even multiple PRMs. Such perfume release may provide a signal as well as a preferred character and intensity; however, the release of only a few PRMs can lead to a less complex scent character or even a polarizing or unpleasant scent experience. This issue can be addressed by balancing the scent character and intensity with PRMs delivered from other TMOT PDTs. For example, the use of a perfume microcapsule (PMC) and a pro-perfume may provide a scent experience that is superior to either PDT that is used individually.

This invention also includes the use of TMOT PDT combinations to provide greater scent intensity and improve scent character at different consumer touch points. For example, the TMOT PDT combination may provide desirable scent soon after the situs is dry, and also at much later time points. The problem with trying to address the need for sustained perfume release over long time periods is that too much perfume may be needed to achieve the desired results. This is especially the case if the PRMs are volatile and dissipate too quickly or have a high odor detection threshold (ODT) and have to be used at levels that are not practical given the theoretical perfume loading limits of the PDT. This need is addressed through the use of TMOT PDT combinations in which perfume release is triggered at different time points. For example, a polymer-assisted delivery TMOT PDT may provide scent shortly after the situs is dry, and an enzyme-triggered pro-perfume may release perfume from clothes that are stored in a hamper waiting to be laundered. The use of PDT combinations also allows the formulator to hide or suppress portions of the perfume until the release of said perfume components are most needed to provide desired intensity and/or character. Leveraging effective triggers for timed perfume release is one means to achieve this goal, in which the headspace of some PRMs carried by one of the TMOT PDTs is suppressed, until release is initiated by the desired trigger.

Another example of a combination of two or more TMOT PDTs includes a starch-coated perfume-loaded zeolite (PLZ), which may deposit on the situs and begin releasing perfume from the situs immediately or soon after the situs is treated with the product; and a pro-perfume, which may begin releasing PRMs from the situs when exposed to a release trigger. In the case of a light-triggered pro-perfume, also known as a photo-pro-perfume, the trigger is electromagnetic radiation such as light. Other pro-perfume triggers include water, pH change, enzymes, or a shift in an equilibrium due to a change in conditions, for example concentration, such that perfume is released at a rate that compliments or enhances the benefits provided by the other TMOT PDT used in the PDT combination, such as the perfume-loaded zeolite.

Another aspect of the present invention uses one or more PDTs on one situs and one or more PDTs on a different situs. For example, the intensity and/or character of perfume released from perfume microcapsules on fabric may be enhanced by the perfume released from the same or different PDT present on hair or skin, such as another perfume microcapsule or a pro-perfume.

In another aspect of the present invention, any of the above perfume delivery technologies (PDTs) may be used to encapsulate or coat any other of the above perfume delivery technologies. Such encapsulation or coating may serve to enhance the stability of the encapsulated or coated perfume or PDT, or serve to alter the deposition or perfume release profile of either the encapsulated or encapsulating PDT. For examples, a core-shell encapsulation technology may be used to encapsulate a pro-perfume with or without additional encapsulated free perfume. In another example, a cyclodextrin molecule or a perfume-loaded cyclodextrin may be encapsulated by a polymer-assisted delivery system in the form of a matrix or reservoir system. Perfume-loaded inorganic zeolites and/or nanotubules may be encapsulated with a polymer matrix or polymer shell. In yet another example, a mixture of perfume and silicone is encapsulated by a melamine-formaldehyde-based polymer system, or a perfume-loaded melamine-formaldehyde-based polymer system is coated with a silicone and/or a perfume-containing silicone. In still another example, a PAD is coated with a PAD, for example, as described in U.S. Pat. No. 5,188,753.

In another aspect of the present invention, a hot melt may be loaded with or used to coat or encapsulate any of the above perfume delivery technologies (PDTs). Such PDT combinations are particular useful for enhancing the consumer experience at the FMOT.

### Examples

A variety of detergent compositions are prepared having the compositions shown in the following examples. In these examples the abbreviated component identifications have the following meanings:
LAS: Sodium linear C₁₂ alkyl benzene sulphonate
CFAA: C₁₂-C₁₄ alkyl N-methyl glucamide
HEDP: Hydroxyethane dimethylene phosphonic acid
DETPMP: Diethylene triamine penta (methylene phosphonic acid), marketed by Monsanto under the Tradename Dequest 2060
TEPAE: Tetreaethylenepentaamine ethoxylate
PVP: Polyvinylpyrrolidone polymer
PVNO: Polyvinylpyridine-N-Oxide, with an average molecular weight of 50,000.
Brightener Disodium 4,4′-bis(2-sulphostyryl)biphenyl and/or Disodium 4,4′-bis(4-anilino-6-morpholino-1.3.5-triazin-2-yl) stilbene-2:2′-disulfonate.
Suds Suppressor-25% paraffin wax Mpt 50° C., 17% hydrophobic silica, 58% paraffin oil Granular suds suppressors 12% Silicone/silica, 18% stearyl alcohol, 70% starch in granular form
PEI Polyethyleneimine
Enzymes: Protease, amylase, cellulase and/or lipase
SRP: Anionically end capped polyesters
MEA Monoethanolamine
SCS Sodium Cumene Sulfonate

A liquid detergent composition containing the perfume delivery system has the following formula:

### Example #1

### Heavy Duty Liquid Laundry Detergent Composition

| Ingredient (% by weight) | A | B | C | D | E | F | G |
|---|---|---|---|---|---|---|---|
| Trisodium Citrate | 4.15 | 2.80 | 2.96 | 3.48 | 2.77 | 3.48 | 3.66 |
| C₁₂₋₁₈ Real Soap | 6.77 | 3.02 | 2.75 | 3.24 | 3.24 | 2.19 | 5.12 |
| Ethanol | 0.85 | 2.22 | 4.02 | 2.59 | 2.22 | 2.50 | 2.50 |
| Monoethanolamine | 1.22 | 1.95 | 2.55 | 1.50 | 5.04 | 1.50 | 1.50 |
| Calcium Formate | 0.03 | 0.01 | 0.08 | 0.05 | 0.30 | 0.06 | 0.04 |
| Propylene Glycol | 5.66 | 2.22 | 2.59 | 4.44 | 5.90 | 4.25 | 1.75 |
| Sodium Formate | 0.05 | 0.25 | 0.088 | 0.103 | 0.125 | 0.15 | 0.35 |
| Borax Premix (38%) | 2.5 | 1.5 | 1.2 | 1.5 | 1 | 3.5 | 0.5 |
| Glycerin | 2.9 | 2.35 | 2.3 | 2.7 | 2.05 | 0.5 | 4 |
| NaOH | 1.3 | 1.1 | 0.88 | 0.837 | 0.95 | 0.25 | 2.25 |
| Hydrophilic Dispersant | 0.25 | 0.44 | 0.55 | 0.65 | 0.86 | 0.15 | 0.96 |
| (PEI 189 E15-E18) | | | | | | | |
| Protease | 0.031 | 0.034 | 0.0272 | 0.032 | 0.023 | 0.015 | 0.051 |
| Cellulase | 0.0008 | 0.001 | 0.0009 | 0.001 | 0.001 | 0.0015 | 0.003 |
| Mannanase | 0.005 | 0.004 | 0.0034 | 0.004 | 0.003 | 0.003 | 0.004 |
| Amylase | 0.0035 | 0.0031 | 0.0025 | 0.003 | 0.0032 | 0.0026 | 0.0018 |
| Suds Suppressor | 0.01 | 0.01 | 0.01 | 0.01 | 0.01 | 0.01 | 0.01 |
| DTPA | 0.11 | 0.15 | 0.13 | 0.15 | 0.19 | 0.22 | 0.09 |
| Hydrophobic Dispersant | 1.21 | 1.19 | 1.09 | 1.29 | 1.75 | 2.4 | 0.75 |
| (PEI 600 E20) | | | | | | | |
| Brightener | 0.106 | 0.125 | 0.106 | 0.125 | 0.125 | 0.106 | 0.125 |
| C₁₂₋₁₄ Alkyl Dimethyl Amine | 0.9 | 1.4 | 0.62 | 0.74 | 0.7 | 0.62 | 0.5 |
| Oxide (Amine Oxide) | | | | | | | |
| C₁₂₋₁₃ AE9 | 2.2 | 2.22 | 1.88 | 2.22 | 2.55 | 2.88 | 4.05 |
| C₂₅AE1.1S Na Paste | 14.44 | 15.75 | 13.06 | 15.37 | 10.25 | 15.24 | 13.2 |
| NaLAS | 6.948 | 5.532 | 4.03 | 4.743 | 6.948 | 5.53 | 4.76 |
| Red HP Liquitint Dye | 0.002 | 0.002 | 0.002 | 0.002 | 0.002 | 0.002 | 0.002 |
| PAD Reservoir System (melamine- | 1.0 | — | — | 0.7 | 2.1 | 0.4 | 0.2 |
| formaldehyde resin PMC) | | | | | | | |
| PAD Matrix System (SAD) | — | — | 2 | — | — | — | — |
| PAD Matrix System (Nanolatex) | — | 0.6 | — | — | — | — | — |
| Amine-Assisted Delivery | — | — | — | 0.2 | 0.2 | — | 0.2 |
| Pro-Perfume | — | — | — | — | — | 0.5 | — |
| ZIC (Inorganic nanotubules) | — | 1.7 | — | — | — | — | — |
| Low KI Perfume Accord | — | — | — | — | — | 0.1 | — |
| Additional Perfume | 0.7 | 0.3 | 0.5 | 0.7 | 0.4 | 0.9 | 0.2 |
| Miscellaneous and water* | | | | | | | |
| PAD Hot Melt** | Yes | — | Yes | — | Yes | — | Yes |
| PAD Perfumed Plastic** | — | Yes | — | — | — | — | Yes |

*Balance
**The products above are packaged in a package comprising a container comprising a cap. The packaging in one aspect comprises the aforementioned PAD matrix system in the form of a Hot Melt adhesive or perfumed plastic. The PAD in form of hot melt adhesive in above examples is placed under or in close proximity to said cap.

### Example #2

### Heavy Duty Liquid Laundry Detergent Composition

| Ingredient (% by weight) | A | B | C | D | E | F | G |
|---|---|---|---|---|---|---|---|
| LAS | 11.5 | 9.0 | — | 4.0 | — | 11.5 | 9.0 |
| C₂₅E2.5S | — | 3.0 | 18.0 | — | 16.0 | — | 3.0 |
| C₄₅E2.25S | 11.5 | 3.0 | — | 16.0 | — | 11.5 | 3.0 |
| C₂₃E9 | — | 3.0 | 2.0 | 2.0 | 1.0 | — | 3.0 |
| C₂₃E7 | 3.2 | — | — | — | — | 3.2 | — |
| CFAA | — | — | 5.0 | — | 3.0 | — | — |
| Top Palm Kernel Fatty Acid | 2.0 | — | 2.0 | 0.5 | 2.0 | 2.0 | — |
| Citric (50%) | 6.5 | 1.0 | 2.5 | 4.0 | 2.5 | 6.5 | 1.0 |
| Ca and/or Ca formate | 0.6 | 0.7 | 0.2 | 0.05 | 0.05 | 0.6 | 0.7 |
| SCS | 4.0 | 1.0 | 3.0 | 1.2 | — | 4.0 | 1.0 |
| Borate | 0.6 | — | 3.0 | 2.0 | 3.0 | 0.6 | — |
| Na hydroxide | 6.0 | 2.0 | 3.5 | 4.0 | 3.0 | 6.0 | 2.0 |
| Ethanol | 2.0 | 1.0 | 4.0 | 4.0 | 3.0 | 2.0 | 1.0 |
| 1,2 Propanediol | 3.0 | 2.0 | 8.0 | 8.0 | 5.0 | 3.0 | 2.0 |
| Monoethanolamine | 3.0 | 1.5 | 1.0 | 2.5 | 1.0 | 3.0 | 1.5 |
| TEPAE | 2.0 | — | 1.0 | 1.0 | 1.0 | 2.0 | — |
| Enzymes | 0.03 | 0.01 | 0.03 | 0.02 | 0.02 | 0.03 | 0.01 |
| SRP | 0.2 | — | 0.1 | — | — | 0.2 | — |
| DTPA | — | — | 0.3 | — | — | — | — |
| PVNO | — | — | 0.3 | — | 0.2 | — | — |
| Brightener | 0.2 | 0.07 | 0.1 | — | — | 0.2 | 0.07 |
| Suds suppressor | 0.04 | 0.02 | 0.1 | 0.1 | 0.1 | 0.04 | 0.02 |
| PAD Reservoir System | 0.5 | 1.4 | 0.2 | 0.6 | 1.0 | — | — |
| PAD Matrix System | — | — | 0.5 | 0.2 | 0.8 | — | 1.0 |
| Amine-Assisted Delivery | 0.1 | — | — | — | — | 0.4 | — |
| Pro-Perfume | — | — | — | 0.2 | 0.2 | 0.6 | 0.3 |
| ZIC (Perfume-Loaded Tubules) | 1.2 | 0.4 | — | — | — | — | — |
| Low KI Perfume Accord | — | — | — | — | 0.2 | 0.1 | 0.1 |
| Additional Perfume | 0.3 | 0.4 | 0.5 | 0.2 | 0.7 | 0.05 | — |
| Miscellaneous and water* | | | | | | | |
| PAD Hot Melt** | — | Yes | — | — | Yes | — | — |
| PAD Perfumed Plastic** | — | — | Yes | — | — | — | Yes |

*Balance
**The products above are packaged in a package comprising a container comprising a cap. The packaging in one aspect comprises the aforementioned PAD matrix system in the form of a Hot Melt adhesive or perfumed plastic.

### Example #3

### Heavy Duty Liquid Laundry Detergent Composition

| Ingredient (% by weight) | A | B | C | D | E | F | G |
|---|---|---|---|---|---|---|---|
| C₁₂₋₁₅ alkyl ether (2.5) sulfate | 17.0 | 15.0 | 21.0 | 19.0 | 15.0 | 19.5 | 24.0 |
| C₁₂₋₁₃ alkyl ethoxylate (9.0) | 2.50 | 2.22 | 2.75 | 2.00 | 1.75 | 2.10 | 1.50 |
| C₁₂₋₁₄ glucose amide | 3.20 | 3.10 | 3.90 | 3.50 | 2.88 | 4.55 | 3.55 |
| Citric Acid | 2.55 | 3.10 | 3.60 | 3.00 | 3.20 | 3.20 | 3.80 |
| C₁₂₋₁₄ Fatty Acid | 1.80 | 2.20 | 2.10 | 2.00 | 2.20 | 2.10 | 2.60 |
| MEA to provide pH of: | 8.0 | 8.3 | 8.5 | 7.8 | 8.0 | 8.0 | 8.1 |
| Ethanol | 3.47 | 3.22 | 1.95 | 3.41 | 3.75 | 4.75 | 3.00 |
| Propanediol | 6.00 | 6.25 | 5.15 | 6.51 | 6.55 | 4.51 | 7.50 |
| Borax | 2.0 | 3.0 | 2.5 | 2.5 | 2.0 | 3.0 | 2.5 |
| PEI - Lupasol G (MW-100) | 0.001 | 0.001 | 0.001 | 0.001 | 0.001 | 0.001 | 0.001 |
| Damascene | 0.01 | — | 0.01 | 0.01 | 0.01 | — | 0.01 |
| Dispersant | 1.15 | 1.00 | 1.50 | 1.18 | 1.15 | 1.00 | 1.50 |
| Na Toluene Sulfonate | 2.11 | 2.75 | 2.50 | 2.50 | 2.25 | 2.75 | 2.50 |
| PAD Reservoir System (PMC) | 0.8 | 0.4 | 1.4 | 0.3 | 1.0 | — | — |
| PAD Matrix System | 0.4 | 1.0 | — | — | 0.2 ^{m} | — | 0.6 |
| Amine-Assisted Delivery | 0.1 | — | — | — | — | 0.2 | — |
| Pro-Perfume | — | — | — | 0.1 | — | 0.4 | 0.5 |
| Perfume-Loaded Tubules | — | — | 0.6 | — | — | — | — |
| Low KI Perfume Accord | — | — | — | — | 0.2 | 0.1 | 0.1 |
| Additional Perfume | 0.7 | 0.7 | 0.5 | 0.7 | 0.7 | 0.5 | — |
| Dye, Brighteners, Enzymes, | | | | | | | |
| Preservatives, Suds Suppressor, | | | | | | | |
| Other Minors, Water* | | | | | | | |
| PAD Hot Melt** | — | Yes | — | — | Yes | — | — |
| PAD Perfumed Plastic** | — | — | Yes | — | — | — | Yes |

*Balance
**The products above are packaged in a package comprising a container comprising a cap. The packaging in one aspect comprises the aforementioned PAD matrix system in the form of a Hot Melt adhesive or perfumed plastic. The PAD in form of hot melt adhesive in above examples is placed under or in close proximity to said cap.
^{m} PAD Matrix System comprising a Silicone-Assisted Delivery (SAD) system

### Example #4

### Fabric Enhancer Composition

| Ingredient (% by weight) | A | B | C | D | E | F | G |
|---|---|---|---|---|---|---|---|
| Fabric Softener Active^{a} | 14.3 | 16.5 | 14.3 | 12.6 | 12.2 | 16.3 | 12.9 |
| Fabric Softener Active^{b} | — | — | — | — | — | — | 4.45 |
| Fabric Softener Active^{c} | — | — | — | — | 4.15 | — | — |
| Ethanol | 2.18 | 2.57 | 2.18 | 1.95 | 1.95 | 2.57 | 2.57 |
| Isopropyl Alcohol | — | — | — | — | — | — | — |
| Starch^{d} | 1.25 | 1.47 | 2.00 | 1.25 | — | 2.30 | 2.30 |
| Formaldehyde Scavenger^{e} | 0.40 | 0.13 | 0.065 | 0.25 | 0.03 | 0.030 | 0.030 |
| Phase Stabilizing Polymer^{f} | 0.21 | 0.25 | 0.21 | 0.21 | 0.14 | — | — |
| Suds Suppressor^{g} | — | — | — | — | — | — | — |
| Calcium Chloride | 0.15 | 0.176 | 0.15 | 0.15 | 0.30 | 0.176 | 0.176 |
| DTPA^{h} | 0.017 | 0.017 | 0.017 | 0.017 | 0.007 | 0.007 | 0.007 |
| Preservative (ppm)^{i, j} | 5 | 5 | 5 | 5 | 5 | 5 | 5 |
| Antifoam^{k} | 0.015 | 0.018 | 0.015 | 0.015 | 0.015 | 0.015 | 0.015 |
| Dye (ppm) | 40 | 40 | 40 | 40 | 40 | 40 | 40 |
| Ammonium Chloride | 0.100 | 0.118 | 0.100 | 0.100 | 0.115 | 0.115 | 0.115 |
| HCl | 0.012 | 0.014 | 0.012 | 0.012 | 0.028 | 0.028 | 0.028 |
| Structurant¹ | 0.01 | 0.01 | 0.01 | 0.01 | 0.01 | 0.01 | 0.01 |
| PAD Reservoir (Perfume Microcapsule) | 0.4 | 0.7 | 0.4 | 0.5 | 0.3 | 0.6 | — |
| PAD Matrix System | 0.5 | 0.6 | 1.0 | — | — | 0.2^{m} | — |
| Amine-Assisted Delivery | 0.2 | — | — | — | — | — | — |
| Pro-Perfume | — | — | — | 0.2 | — | — | 0.6 |
| ZIC (Perfume-Loaded Tubules) | — | — | — | — | 0.8 | — | — |
| Low KI Perfume Accord | 0.1 | 0.1 | — | — | 0.2 | — | 0.1 |
| Additional Perfume | 0.3 | 0.7 | 0.5 | 0.4 | 0.3 | 0.3 | 0.9 |
| Deionized Water* | | | | | | | |
| PAD Hot Melt** | — | Yes | — | — | — | — | — |
| PAD Perfumed Plastic** | — | — | Yes | — | — | — | Yes |

*Balance
^{a}N,N-di(tallowoyloxyethyl)-N,N-dimethylammonium chloride.
^{b}Methyl bis(tallow amidoethyl)2-hydroxyethyl ammonium methyl sulfate.
^{c}Reaction product of Fatty acid with Methyldiethanolamine in a molar ratio 1.5:1, quaternized with Methylchloride, resulting in a 1:1 molar mixture of N,N-bis(stearoyl-oxy-ethyl) N,N-dimethyl ammonium chloride and N-(stearoyl-oxy-ethyl) N,-hydroxyethyl N,N dimethyl ammonium chloride.
^{d}Cationic high amylose maize starch available from National Starch under the trade name CATO ®.
^{e}The formaldehyde scavenger is as described in the art.
^{f}Copolymer of ethylene oxide and terephthalate having the formula described in U.S. Pat. No. 5,574,179 at col.15, lines 1-5, wherein each X is methyl, each n is 40, u is 4, each R1 is essentially 1,4-phenylene moieties, each R2 is essentially ethylene, 1,2-propylene moieties, or mixtures thereof.
^{g}SE39 from Wacker
^{h}Diethylenetriaminepentaacetic acid.
ⁱKATHON ® CG available from Rohm and Haas Co. “PPM” is “parts per million.”
^{j}Gluteraldehyde
^{k}Silicone antifoam agent available from Dow Corning Corp. under the trade name DC2310.
^{l}Hydrophobically-modified ethoxylated urethane available from Rohm and Haas under the Tradename Aculan 44.
^{m}PAD Matrix System comprising a Silicone-Assisted Delivery (SAD) system
**The products of Examples 1-4 above are packaged in a package comprising a container comprising a cap. The packaging comprises any aforementioned PAD in form of Perfumed Plastic insert and the cap comprises any aforementioned PAD in form of Hot Melt. The PAD in form of Hot Melt is typically placed under or in close proximity to said cap.
**The products of Examples 5-6 below are packaged in a package comprising a container comprising a cap or a lid. The packaging comprises any aforementioned PAD in form of Perfumed Plastic insert and the lid comprises any aforementioned PAD in form of Hot Melt. The PAD in form of Hot Melt, for example, may be placed under the lid, on the box, on the handle, on the label, etc. The Perfumed Plastic may also be made to be part of the package.

### Example #5

### Granular Detergent Composition

A heavy duty granular detergent (HDG) composition is prepared containing the perfume delivery system. Such a granular detergent composition has the following formula:

| Ingredient (% by weight) | A | B | C | D | E | F | G |
|---|---|---|---|---|---|---|---|
| C₁₂ Linear alkyl benzene sulfonate | 8.25 | 7.75 | 9.55 | 9.31 | 8.53 | 10.22 | 9.22 |
| C₁₄₋₁₅ alkyl sulfonate | 12.15 | 15.15 | 13.55 | 12.74 | 9.50 | 13.25 | 12.00 |
| Zealite Builder | 26.34 | 23.66 | 29.62 | 27.79 | 30.15 | 28.25 | 29.39 |
| Sodium Carbonate | 29.35 | 26.33 | 27.11 | 27.31 | 28.28 | 29.35 | 27.35 |
| PEG 4000 | 1.55 | 1.43 | 1.87 | 1.60 | 1.96 | 1.55 | 1.63 |
| Dispersant | 2.23 | 2.65 | 1.98 | 2.26 | 2.24 | 2.26 | 2.21 |
| C₁₂₋₁₃ alkyl ethoxylate (E9) | 1.4 | 1.3 | 1.6 | 1.5 | 1.8 | 1.3 | 1.4 |
| Sodium Perborate | 1.01 | 1.23 | 1.73 | 1.03 | 1.01 | 0.96 | 0.89 |
| Soil Release Polymer | 0.40 | 0.22 | 0.35 | 0.41 | 0.40 | 0.55 | 0.85 |
| Enzymes | 0.54 | 0.58 | 0.53 | 0.59 | 0.78 | 0.35 | 0.89 |
| PAD Reservoir System | — | 0.8 | — | 1.1 | 0.4 | 0.6 | — |
| PAD Matrix System | — | 0.6 | — | — | — | — | 0.2 |
| Starch-Encapsulated Accord | 0.5 | — | 0.2 | 0.1 | — | — | 0.2 |
| ZIC (Perfume-Loaded Zeolite) | 0.5 | — | — | — | — | 0.3 | — |
| ZIC (Nanotubules) | — | — | — | — | 0.7 | — | — |
| Beta-Cyclodextrin Derivative | — | — | — | — | — | — | 0.2 |
| Pro-Perfume | — | — | 0.1 | 0.02^{p} | 0.2^{q} | — | 0.1^{r} |
| Additional Perfume | 0.5 | 0.2 | 0.1 | 0.1 | 0.1 | 0.3 | — |
| Amine-Assisted Delivery | — | — | — | — | — | — | 0.1 |
| Brightener, Suds Suppressor, Other | | | | | | | |
| Minors, and Moisture* | | | | | | | |
| PAD Hot Melt** | — | Yes | — | — | Yes | — | — |
| PAD Perfumed Plastic** | — | — | Yes | — | — | — | Yes |

*Balance
^{p}Pro-perfume comprising an Amine Reaction Product (ARP) System based on PEI
^{q}Pro-perfume comprising a nitrogen-based photo-pro-perfume (PPP) System
^{r}Pro-perfume is a 3′,5′-dimethoxybenzoin derivative that releases a perfume alcohol.

### Example #6

### Granular Detergent Composition

| Ingredient | | | | | | | |
|---|---|---|---|---|---|---|---|
| (% by weight) | A | B | C | D | E | F | G |
| C₁₂ Linear alkyl | 8.25 | 7.75 | 9.55 | 9.31 | 8.53 | 10.22 | 9.22 |
| benzene sulfonate | | | | | | | |
| C₁₄₋₁₅ alkyl sulfonate | 12.15 | 15.15 | 13.55 | 12.74 | 9.50 | 13.25 | 12.00 |
| Zeolite Builder | 26.34 | 23.66 | 29.62 | 27.79 | 30.15 | 28.25 | 29.39 |
| Sodium Carbonate | 29.35 | 26.33 | 27.11 | 27.31 | 28.28 | 29.35 | 27.35 |
| PEG 4000 | 1.55 | 1.43 | 1.87 | 1.60 | 1.96 | 1.55 | 1.63 |
| Dispersant | 2.23 | 2.65 | 1.98 | 2.26 | 2.24 | 2.26 | 2.21 |
| C₁₂₋₁₃ alkyl ethoxylate | 1.4 | 1.3 | 1.6 | 1.5 | 1.8 | 1.3 | 1.4 |
| (E9) | | | | | | | |
| Sodium Perborate | 1.01 | 1.23 | 1.73 | 1.03 | 1.01 | 0.96 | 0.89 |
| Soil Release Polymer | 0.40 | 0.22 | 0.35 | 0.41 | 0.40 | 0.55 | 0.85 |
| Enzymes | 0.54 | 0.58 | 0.53 | 0.59 | 0.78 | 0.35 | 0.89 |
| PAD in form of Matrix | — | — | 0.8^{m} | — | 0.3 | — | — |
| System | | | | | | | |
| Starch-Encapsulated | 0.5 | — | — | 0.2 | — | — | 0.3 |
| Accord | | | | | | | |
| Amine-Reaction | — | — | — | 0.05 | — | — | 0.1 |
| Product | | | | | | | |
| ZIC (Perfume-Loaded | — | — | — | — | — | — | 0.2 |
| Zeolite) | | | | | | | |
| Cyclodextrin | 0.3 | — | — | — | — | — | — |
| Pro-Perfume | — | 0.4 | 0.1 | — | — | 0.3 | — |
| ZIC (Perfume-Loaded | — | — | — | — | 0.5 | — | — |
| Tubules) | | | | | | | |
| Additional Spray-On | 0.2 | 0.3 | — | 0.2 | 0.4 | — | 0.1 |
| Perfume | | | | | | | |
| Damascone | — | — | 0.01 | 0.01 | — | — | — |
| Amine-Assisted | — | — | — | 0.08 | — | — | — |
| Delivery | | | | | | | |
| Brightener, Suds | | | | | | | |
| Suppressor, Other | | | | | | | |
| Minors, and Moisture* | | | | | | | |
| PAD Hot Melt** | — | Yes | Yes | Yes | — | — | — |
| PAD Perfumed | — | Yes | — | — | Yes | Yes | Yes |
| Plastic** | | | | | | | |

*Balance
^{m}PAD Matrix System comprising a Silicone-Assisted Delivery (SAD) system

### Example #7

### An Apparatus in the Form of a Non-Woven Pad Used in Conjunction with a Hard Surface Cleaning Composition

The following are non-limiting examples of hard surface cleaning compositions that are useful in the present invention, especially in combination with cleaning pads and/or cleaning implements described in U.S. Pat. No. 6,663,306 B2.

| Ingredient (% by weight) | A | B | C | D | E |
|---|---|---|---|---|---|
| Neodol 1-5¹ | 0.03 | — | 0.03 | — | — |
| Witconate NAS-8² | 0.01 | 0.02 | 0.01 | — | — |
| Planteran 2000³ | — | 0.05 | — | 0.004 | 0.004 |
| Ammonia Hydroxide | — | — | — | 0.1 | — |
| Glacial Acetic Acid | — | — | — | — | — |
| DMAMP-80⁴ | 0.01 | 0.01% | 0.06% | — | 0.01% |
| Dowanol PnP⁵ | 2.0 | 2.0 | 2.0 | 4.0 | 4.0 |
| Polyvinvylpyridine N-oxide | 0.015 | 0.015 | 0.015 | 0.003 | 0.003 |
| 1-Methoxy-2-Butanol | — | — | — | — | — |
| Silicone suds suppressor⁶ | 0.00125 | 0.00125 | 0.00125 | — | — |
| Perfume | 0.033 | 0.06 | 0.035 | — | — |
| Xylenolphthalein | — | — | 0.001 | — | — |
| PAD Reservoir System | 0.19 | 0.39 | — | — | 0.25 |
| PAD Matrix System | 0.05 | 0.1 | 0.45 | — | — |
| Amine-Assisted Delivery | 0.08 | — | — | — | — |
| Pro-Perfume | — | — | 0.07 | 0.10 | — |
| ZIC (Perfume-Loaded Tubule) | — | — | — | — | 0.42 |
| Low KI Perfume Accord | 0.004 | 0.01 | — | — | 0.02 |
| Deionized water | Balance | Balance | Balance | Balance | Balance |
| PAD Hot Melt** | Yes | — | Yes | Yes | — |
| PAD Perfumed Plastic** | — | — | — | — | Yes |
| Ingredient (% by weight) | F | G | H | I | J |
| Neodol 1-5¹ | — | 0.03 | 0.03 | 0.03 | 0.03 |
| Witconate NAS-8² | — | 0.01 | 0.01 | 0.01 | 0.01 |
| Planteran 2000³ | 0.004 | — | — | — | — |
| Ammonia Hydroxide | 0.01 | — | — | — | — |
| Glacial Acetic Acid | — | 0.05 | 0.05 | — | 0.05 |
| DMAMP-80⁴ | — | — | — | 0.01 | — |
| Dowanol PnP⁵ | 4.0 | — | 2.0 | — | — |
| Polyvinvylpyridine N-oxide | 0.003 | 0.015 | 0.015 | 0.015 | 0.015 |
| 1-Methoxy-2-Butanol | — | — | — | 2.0 | — |
| Silicone suds suppressor⁶ | — | 0.00125 | 0.00125 | 0.00125 | 0.00125 |
| Perfume | 0.015 | 0.03 | 0.03 | 0.03 | 0.03 |
| Xylenolphthalein | — | — | — | — | — |
| PAD Reservoir System | 0.18 | 0.36 | 0.13 | — | — |
| PAD Matrix System | — | — | — | — | — |
| Amine-Assisted Delivery | — | — | — | 0.08 | 0.10 |
| Light-Triggered Pro-Perfume | — | — | 0.22 | 0.11 | 0.10 |
| Additional Low KI Perfume Accord | — | 0.015 | — | — | 0.11 |
| Deionized water | Balance | Balance | Balance | Balance | Balance |
| PAD Hot Melt** | Yes | — | Yes | Yes | — |
| PAD Perfumed Plastic** | — | — | — | — | Yes |

¹C₁₁E₅ alcohol ethoxylate commercially available from Shell Chemical.
²Linear C₈ sulfonate commercially available from Witco Chemical.
³C₈-C₁₆ alkyl polyglucoside commercially available from Henkel.
⁴2-dimethylamino-2-methyl-1-propanol commercially available from Angus Chemical.
⁵Propylene glycol n-propyl ether commercially available from Dow Chemical.
⁶Silicone suds suppressor commercially available from Dow Coming under the trade name Dow Corning AF ® Emulsion.

For Example #7, the apparatus, in the form of a non-woven pad used in conjunction with the above hard surface cleaning compositions, may also optionally contain combinations of perfume delivery technologies including but not limited to those described below. Any combination of PDTs in the solution composition may optionally be combined with any combination of PDTs in the non-woven pad composition.

| Ingredient (% by weight) | K | L | M | N | O |
|---|---|---|---|---|---|
| Apparatus in the form of a non-woven | >90 | >90 | >90 | >90 | >90 |
| pad as described in US 06663306 | | | | | |
| Perfume | 0.05 | — | — | — | 0.1 |
| Low KI Perfume Accord | 0.02 | — | — | — | — |
| PAD Reservoir System | 0.2 | 0.03 | — | 0.1 | — |
| PAD Matrix System | — | 0.01 | 0.02 | 0.15^{m} | — |
| Starch-Encapsulated Accord | — | — | 0.058 | — | — |
| Amine-Reaction Product | — | — | — | 0.018 | — |
| ZIC | — | — | — | — | 0.01 |
| Cyclodextrin | — | — | — | — | — |
| Amine-Assisted Delivery | 0.01 | — | — | — | — |
| Pro-Perfume (ARP) | — | — | — | 0.09^{p} | — |
| Pro-Perfume (PPP) | — | 0.09^{q} | — | | — |
| Other minors and moisture | Balance | Balance | Balance | Balance | Balance |

^{m}PAD Matrix System comprising a Silicone-Assisted Delivery (SAD) system
^{p}Pro-perfume comprising an Amine Reaction Product (ARP) System based on PEI
^{q}Pro-perfume comprising a nitrogen-based photo-pro-perfume (PPP) System

| Ingredients (% by weight) | P | Q | R | S | T |
|---|---|---|---|---|---|
| Apparatus in the form of a | >90 | >90 | >90 | >90 | >90 |
| non-woven pad as | | | | | |
| described in US | | | | | |
| 06663306 | | | | | |
| Perfume | 0.04 | 0.01 | — | — | 0.07 |
| PAD Reservoir System in | 1.9 | 4.0 | 0.3 | — | — |
| form of PMC | | | | | |
| PAD Matrix System in | 0.5 | — | — | 1.4 | — |
| form of SAD | | | | | |
| Starch-Encapsulated | — | — | 0.55 | — | — |
| Accord | | | | | |
| ZIC in form of fused silica | — | — | — | 0.6 | — |
| Cyclodextrin | — | — | — | — | 0.75 |
| Amine-Assisted Delivery | — | 0.2 | — | — | — |
| Pro-Perfume | — | — | — | — | — |
| Other minors and moisture* | | | | | |
| PAD Hot Melt** | Yes | — | — | — | — |
| PAD Perfumed Plastic** | — | — | — | Yes | — |

*Balance
**The packaging in one aspect comprises the aforementioned PAD matrix system in the form of a Hot Melt adhesive or perfumed plastic.

### Example #8

In one aspect of the present invention, the perfume delivery system is particular useful for heavy duty liquid detergents and/or fabric enhancers. The perfume delivery system consists of:
1) PAD Matrix System and/or AAD System in the form of a cationic, anionic or nonionic polymer.
2) PAD Reservoir System in the form of a Perfume Microcapsule (PMC)
3) optional free perfume

Without being bound by theory, a perfume delivery technology (PDT) comprising a PAD matrix system and/or an AAD system or systems functions by interacting with perfume to alter the stability, deposition and release of the perfume at the FMOT, SMOT and TMOT. A perfume microcapsule (PMC) also interacts with perfume to alter the stability, deposition and release of the perfume at the FMOT, SMOT and TMOT. Surprisingly, a particularly synergistic benefit is observed when one or more PAD matrix system and/or one or more AAD system is combined with one or more PAD reservoir system in the form of a perfume microcapsule (PMC); the benefit is greater than the benefit observed for either PDT separately or the expected combination of the benefits. In one aspect of the present invention, the PMC may be pre-loaded with perfume, and the polymer of the PAD matrix system may be pre-loaded with perfume or added separately from the perfume to the product. Without being bound by theory, the combination of a PAD matrix system and/or an AAD system with a perfume microcapsule serves one or more functions. One function is that the PAD and/or the AAD PDT(s) increase the deposition of the PMC. A second function is that PAD and/or AAD serve to “scavenge” perfume that may be available due to the presence of the PMC. Without being bound by theory, some perfume may “leak” out of the PMC during any stage of the PMC life cycle, which includes manufacturing, processing, purifying, isolating, shipping, formulating with other ingredients or during storage in the delivery system or product. Without being bound by theory, the PAD and/or AAD PDT is especially effective at improving the delivery of certain PRMs, especially those PRMs with a CLogP less than about 3 and/or those PRMs with a Kovats Index (KI) value less than 1500. Some PRMs may not be effectively held within the PMC, especially during product storage and at high temperature; and such PRMs may be delivered more effectively by combining PMC with PAD and/or AAD PDTs. As such, one may tune the perfume diffusion stability, perfume and PDT situs deposition and perfume release profiles by combining PMC with PAD and/or AAD PDTs to achieve a perfume intensity and character at the FMOT, SMOT and TMOT that may not be achieved without the PDT combination provided. Without being bound by theory, such PRMs are not delivered as effectively when combined with PAD and/or AAD PDTs in the absence of PMC because some of these PRMs are high impact and may have low odor detection thresholds (ODTs). It is therefore typically necessary to use the PMC to suppress the level of free perfume present in the product so that the perfume does not distort the consumer experience at the FMOT. Other PRMs have limited stability during product storage, and thus without being bound by theory, the PAD and/or AAD PDTs serve to minimize further PRM degradation by interacting with those PRMs that are added as free perfume or that may have leaked or diffused out of the PMC or other perfume delivery system(s) over time.

### Example #9

In another aspect of the present invention, the perfume delivery system is particular useful for heavy duty liquid detergents and/or fabric enhancers. The perfume delivery system consists of:
1) PAD Matrix System and/or AAD System in the form of a cationic, anionic or nonionic polymer.
2) PAD Reservoir System in the form of a Perfume Microcapsule (PMC)
3) optional free perfume
4) FMOT PDT “on or in the product or package”
   FMOT PDT: Without being bound by theory, FMOT PDT such as PAD Hot Melt and/or PAD Perfumed Plastic functions by perfume interacting with a perfume carrier, such as a polymer, to alter the perfume chemical stability and perfume release rate to provide a consumer desired intensity and character. Surprisingly, a particularly synergistic benefit is observed when the FMOT PDT is used in combination with PAD and/or AAD PDTs and/or PMC, preferably also with free perfume. Without being bound by theory, the PDT combination serves to mitigate scent character distortion that can occur from the interaction of PAD and/or AAD PDTs and/or PMC with pre-loaded or separately added perfume. With the use of the FMOT PDT, the combination of perfume with PAD and/or AAD and/or PMC PDTs may be optimized in order to provide the greatest consumer benefit during the SMOT and TMOT. It can be difficult for the formulator of perfume and PDTs to achieve the ideal scent intensity and character at all consumer touch points. The use of FMOT PDT surprisingly reduces the impact of the formulated perfume and PDTs on the FMOT experience, such as neat product odor. As such, this minimizes some constraints around perfume formulation and allows the formulator to optimize the PAD and/or AAD and/or PMC PDTs especially for greatest TMOT impact. The distortion in neat product odor that may be needed to maximize TMOT benefit is diminished by this PDT combination. In particularly, the need for FMOT PDT is surprisingly greatest when used in conjunction with the combination of PAD and/or AAD and/or PMC PDTs, due to the unexpected decrease in neat product intensity and change of character associated with the unexpected perfume interactions described above, which is mitigated by the FMOT PDT.

### Example #10

In one aspect of the present invention, the perfume delivery system is particular useful for heavy duty liquid detergents and/or fabric enhancers. In another aspect, the perfume delivery system consists of:
1) (optional) Cationic polymer (PAD and/or AAD)
2) PMC based on urea or melamine formaldehyde
3) Free perfume
4) (optional) Hot melt or perfumed plastic insert on or under the product cap or lid

### Example #11

In one aspect of the present invention, the perfume delivery system is particular useful for heavy duty liquid detergents and/or fabric enhancers. In another aspect, the perfume delivery system consists of:
1) (optional) Cationic polymer (PAD and/or AAD)
2) PMC based on urea or melamine formaldehyde
3) Free perfume
4) (optional) Hot melt or perfumed plastic insert on or under the product cap or lid
5) Photo-Pro-Perfume (PPP)

Without being bound by theory, PPP, PMC, PAD, AAD and APAD PDTs interact with perfume to alter the stability, deposition and release of the perfume at the FMOT, SMOT and TMOT. Surprisingly, a particularly synergistic benefit is observed when PPPs are combined with PMC and/or PAD and/or AAD PDT(s). In particular, the PPP PDT can be selected such that PRM release from the situs is complimentary to that observed for PMC and/or PAD and/or AAD PDTs. In particularly, although PMC and/or PAD and/or AAD PDTs are effective at delivering a broad range of PRMs to the situs, in some cases, having certain PRMs present on the situs at too low a concentration or releasing into the headspace above the situs at too slow a rate will lead to a distortion of the desired intensity and character. This can especially be the case after longer time periods since situs drying. We have surprisingly found that the character and/or intensity can be maintained for longer period of time when PPP is used in combination with PMC and/or PAD PDTs. In addition, the PMC and PAD and/or AAD PDTs can increase the deposition of the PPP PDT. Also without being bound by theory, the PMC and/or PAD and/or AAD PDTs can alter the release profile from the dry situs of the perfume from the PPP. Without being bound by theory, this unexpected result may be due to the other PDTs altering the interaction of the PPP and the released PRMs from the dry situs, thus altering perfume release properties.

### Example #12

In one aspect of the present invention, the perfume delivery system is particular useful for Laundry Granules. In another aspect, the perfume delivery system consists of:
1) SEA; and
2) PMC; and
3) (optional) Spray on perfume; and
4) (optional) FMOT PDT “on or in the product or package”
   Without being bound by theory, it has been surprisingly found that there is a symbiotic effect of using the combination of SEA and PMC. Both PDTs provide improved retention of volatile perfume prior to consumer use; however, the different release profiles for SEA and PMC enable a release system that is superior to using either PDT separately. The release of perfume from SEA upon use of product is relatively fast, whereas the release from PMC is relatively slow. This combination of bloom and triggered release from dry situs provide benefit throughout the use experience.

### Example #13

In one aspect of the present invention, the perfume delivery system is particular useful for Laundry Granules. In another aspect, the perfume delivery system consists of:
1) PLZ and/or PLT; and
2) PMC; and
3) (optional) Spray on perfume; and
4) (optional) FMOT PDT “on or in the product or package”
5) (optional) SEA

Without being bound by theory, it has been surprisingly found that there is a symbiotic effect of using the combination of PLZ and/or PLT and PMC. Both PDTs provide improved retention of volatile perfume prior to consumer use; however, the different release profiles for SEA and PMC, particularly from dry situs, provide a release system that is unexpectedly superior to using either PDT separately. Without wishing to be bound by theory, the perfume release profiles for PLZ and/or PLT and PMC is different. For PLZ, the release is triggered by removal of a water-soluble coating to initiate perfume diffusion into the headspace of the dry situs. For PLT, the release is triggered by removal of any end-capping groups and diffusion also by reverse capillary action. For PMC, the release can also be triggered by the removal of an optional water-soluble coating, and/or by diffusion into the headspace of the dry situs. Each PDT has been optimized to deliver perfume accords that work best with the technology. For PLZ, PRM selection is also made based on molecular size. For PLT, PRM selection is based on a number of factors including volatility, odor detection threshold, size and Kovats Index (KI) value. For PMC the selection includes considerations of wall permeability or leakage. Thus, a combination of these PDTs allows for a fuller palette of PRMs to achieve the desired intensity and character. In addition to the perfume diffusion release mechanism, the PMC can be selected to provide a friction-triggered release or burst of perfume, leading to greater perfume intensity and a preferred character. Such a combination of perfume diffusion of preferred PRMs and a burst of perfume due to alternate release mechanism provides unexpectedly improved benefits over what would be expected.

### Example #14

In one aspect of the present invention, the perfume delivery system is particular useful for Laundry Granules. In another aspect, the perfume delivery system consists of:
1) PDT selected from PLZ, SEA, CD, PMC
2) (optional) Spray on perfume; and
3) FMOT PDT “on or in the product or package”

Laundry granules are difficult to smell because of closed container. FMOT PDT on the outside of the product or package provides FMOT scent intensity and character. In another aspect, the presence of the FMOT PDT on the inside of the package provides unexpected synergies with other PDTs.

### Example #15

In one aspect of the present invention, the perfume delivery system is particular useful for Laundry Granules. In another aspect, the perfume delivery system consists of:
1) Nanotubules (PLT); and
2) Perfume Microcapsule (PMC); and
3) (optional) Spray on perfume; and
4) (optional) FMOT PDT “on or in the product or package”
5) (optional) SEA

### Example #16

In one aspect of the present invention, the perfume delivery system is particular useful for a Fabric Softener Sheet. In another aspect, the perfume delivery system consists of:
1) Cyclodextrin (CD); and
2) Perfume Microcapsule (PMC); and
3) (optional) neat perfume; and
4) (optional) FMOT PDT “on or in the product or package”
5) (optional) SEA

### Example #17

In one aspect of the present invention, the perfume delivery system is particular useful for a Fabric Softener. In another aspect, the perfume delivery system consists of:
1) Hexarose (geranyl palmitate) 0.25%; and
2) Perfume Microcapsule (PMC) 1.3%; and
3) (optional) neat perfume 0.6%; and
4) (optional) FMOT PDT “on or in the product or package”
5) (optional) SEA

All percentages listed in this Example 17 are based on total product weight independent of any container and/or packaging.

The weight percentages of the aforementioned ingredients may be varied as desired, for example, the hexarose (also known as (E)-3,7-dimethyl-2,6-octadienylhexadecanoate) may be present at levels of from about 0.05% to about 10%, from about 0.1% to about 1%, or even from about 0.25% to about 0.75%; the PMC may be present at levels of from about 0.05% to about 10%, from about 0.1% to about 3%, or even from about 0.3% to about 1.5%; and the neat perfume may be present at levels of from about 0.01% to about 10%, from about 0.1% to about 3%, or even from about 0.5% to about 1.5%. The aforementioned perfume delivery system may be used in other consumer products, including other cleaning and/or treatment products, for example such products that contain minimal or no lipase. In addition, other terpene esters may be substituted for hexarose or even beta-keto esters may be used. Suitable terpene esters may be produced in accordance with the teachings of U.S. Pat. No. 5,652,205. For example, hexarose may be made by following the teachings of Example I of U.S. Pat. No. 5,625,205 and replacing the succinic acid with palmitic acid or Example II of U.S. Pat. No. 5,652,205 and replacing lauroyl chloride with palmitoyl chloride. Suitable beta-keto esters may be made in accordance with the teachings of USPs 6,100,233 and 5,965,767.

### Example #18

In one aspect of the present invention, the perfume delivery system is particular useful for a hair shampoo. In another aspect, the perfume delivery system consists of:

| Ingredients (% by weight) | A | B | C | D | E | F | G |
|---|---|---|---|---|---|---|---|
| Ammonium Laureth/Lauryl Sulfate | 16 | 14 | 20 | 16 | 14 | 20 | 16 |
| Glycol Distearate | 1.5 | 1.1 | 1.6 | 1.5 | 1.1 | 1.6 | 1.5 |
| Dimethicone | 1.4 | 1.1 | 1.8 | 1.4 | 1.1 | 1.8 | 1.4 |
| Cetyl Alcohol | 0.90 | 1.2 | 1.4 | 0.90 | 1.2 | 1.4 | 0.90 |
| Cocamide MEA | 0.75 | 0.95 | 0.55 | 0.75 | 0.95 | 0.55 | 0.75 |
| Sodium Chloride | 0.65 | 1.0 | 1.3 | 0.65 | 1.0 | 1.3 | 0.65 |
| Polyquaternium-10 (LR-400) | 0.50 | 0.30 | 0.20 | 0.50 | 0.30 | 0.20 | 0.50 |
| Sodium Citrate | 0.60 | 0.40 | 0.50 | 0.60 | 0.40 | 0.50 | 0.60 |
| Hydrogenated Polydecene | 0.30 | 0.20 | 0.70 | 0.30 | 0.20 | 0.70 | 0.30 |
| Sodium Benzoate | 0.20 | 0.35 | 0.40 | 0.20 | 0.35 | 0.40 | 0.20 |
| Disodium EDTA | 0.12 | 0.085 | 0.15 | 0.12 | 0.085 | 0.15 | 0.12 |
| Trimethylolpropane Tricaprylate/ | 0.10 | 0.15 | 0.10 | 0.10 | 0.15 | 0.10 | 0.10 |
| Tricaprate | | | | | | | |
| Citric Acid | 0.040 | 0.050 | 0.040 | 0.040 | 0.050 | 0.040 | 0.040 |
| Pro-vitamins | 0.060 | — | 0.030 | 0.060 | — | 0.030 | 0.060 |
| Methylchloroisothiazolinone/ | 0.0004 | 0.0010 | 0.0003 | 0.0004 | 0.0010 | 0.0003 | 0.0004 |
| Methylisothiazolinone | 0.0001 | 0.0002 | 0.0003 | 0.0001 | 0.0002 | 0.0003 | 0.0001 |
| PAD Reservoir System in form of a | 0.5 | 1.4 | 0.2 | 0.6 | 1.0 | — | — |
| Perfume Microcapsule (PMC) | | | | | | | |
| PAD Matrix System | — | 0.3^{m} | 0.4 | — | 0.6 | — | 1.1 |
| Amine-Assisted Delivery | 0.2 | — | — | — | — | 0.4 | — |
| Pro-Perfume | — | — | — | — | 0.2 | 0.6 | — |
| Perfume-Loaded Tubules | — | — | — | — | — | — | 0.3 |
| Low KI Perfume Accord | — | — | — | 0.2 | 0.2 | 0.1 | 0.3 |
| Additional Perfume | 0.5 | 0.3 | 0.7 | 0.4 | 0.6 | 1.1 | — |
| Water/Carriers/Aesthetics* | | | | | | | |
| PAD Hot Melt** | — | Yes | — | Yes | Yes | — | — |
| PAD Perfumed Plastic** | — | — | Yes | — | — | — | Yes |

*balance
**Hot Melt and/or Perfumed Plastic on or in package not included in formula composition balance. For this example, FMOT technology is on or under the cap.
^{m}PAD Matrix System comprising a Silicone-Assisted Delivery (SAD) system

### Example #19

In one aspect of the present invention, the perfume delivery system is particular useful for a hair conditioner. In another aspect, the perfume delivery system consists of:

| Ingredients (% by weight) | A | B | C | D | E | F | G |
|---|---|---|---|---|---|---|---|
| Ammonium laureth-3-sulfate | 10 | 10 | 12 | 10 | 10 | 10 | 10 |
| Ammonium lauryl sulface | 10 | 6 | 4 | 6 | 6 | 10 | 6 |
| Cocamide MEA | 1.45 | 0.85 | 0.68 | 0.8 | 0.8 | 1.45 | 0.85 |
| Polyquat 10⁵ | 0.2 | 0 | 0.4 | 0 | 0.15 | 0.2 | 0 |
| Guar hydroxypropyltrimonium chloride⁶ | 0 | 0.2 | 0 | 0.15 | 0 | 0 | 0.2 |
| Hydrogenated Polyalpha Olfefin⁷ | 0.4 | 0.32 | 0.25 | 0.4 | 0.3 | 0.4 | 0.32 |
| Cetyl alcohol | 0.42 | 0 | 0.42 | 0.6 | 0.42 | 0.42 | 0 |
| Stearyl alcohol | 0.18 | 0 | 0.18 | 0 | 0.18 | 0.18 | 0 |
| Ethylene glycol distearate | 1.5 | 2.0 | 1.5 | 1.5 | 2.0 | 1.5 | 2.0 |
| Dimethicone⁸ | 2.0 | 1.0 | 0 | 0 | 1.5 | 2.0 | 1.0 |
| Monosodium Phosphate | 0.1 | 0.1 | 0.1 | 0 | 0 | 0.1 | 0.1 |
| Disodium Phosphate | 0.1 | 0.2 | 0.2 | 0 | 0 | 0.1 | 0.2 |
| EDTA | 0.1 | 0.1 | 0.1 | 0.1 | 0.1 | 0.1 | 0.1 |
| DMDM Hydantoin | 0.37 | 0.37 | 0.37 | 0.37 | 0.37 | 0.37 | 0.37 |
| PAD in form of Microcapsule | 0.5 | 1.4 | 0.2 | 0.6 | 1.0 | — | — |
| PAD in form of Matrix System | — | 0.3 | 0.4 | — | 0.6 | — | 1.1 |
| Amine-Assisted Delivery | 0.2 | — | — | — | — | 0.4 | — |
| Pro-Perfume | — | — | — | — | 0.2 | 0.6 | — |
| Perfume-Loaded Tubules | — | — | — | — | — | — | 0.3 |
| Low KI Perfume Accord | — | — | — | 0.2 | 0.2 | 0.1 | 0.3 |
| Additional Perfume | 0.45 | 0.3 | 0.6 | 0.45 | 0.6 | 1.1 | — |
| Water/Carriers/Aesthetics⁹ | | | | | | | |
| PAD Hot Melt¹⁰ | — | Yes | — | Yes | Yes | — | — |
| PAD Perfumed Plastic¹⁰ | — | — | Yes | — | — | — | Yes |

⁵JR 30M available from Amerchol
⁶Jaguar C-17 available from Rhone-Poulenc
⁷SHF 62 available from Mobil Chemical
⁸Dimethicone is a 40(gum)/60(fluid) weight ratio blend of SE-76 dimethicone gum available from General Electric Silicones Division and a dimethicone fluid having a viscosity of 350 centistoke.
⁹Balance
¹⁰Hot Melt and/or Perfumed Plastic on or in package not included in formula composition balance. For this example, FMOT technology is on or under the cap.

### Example #20

In one aspect of the present invention, the perfume delivery system is particular useful for a body wash. In another aspect, the perfume delivery system consists of:

| Ingredients (% by weight) | A | B | C | D | E | F | G |
|---|---|---|---|---|---|---|---|
| Sodium Laureth Sulfate | 7.5 | 8.5 | 8.2 | 7.5 | 8.5 | 8.2 | 7.5 |
| Cocamidopropyl Betaine | 6.5 | 5.5 | 4.5 | 6.5 | 5.5 | 4.5 | 6.5 |
| Sodium Lauroyl Sarcosinate | 0.75 | 0.65 | 1.2 | 0.75 | 0.65 | 1.2 | 0.75 |
| Citric Acid | 0.26 | 0.33 | 0.38 | 0.26 | 0.33 | 0.38 | 0.26 |
| Guar Hydroxypropyltrimonium Chloride | 0.50 | 0.30 | 0.30 | 0.50 | 0.30 | 0.30 | 0.50 |
| Lauryl Alcohol | 0.65 | 0.80 | 0.77 | 0.65 | 0.80 | 0.77 | 0.65 |
| DMDM Hydantoin | 0.21 | 0.26 | 0.11 | 0.21 | 0.26 | 0.11 | 0.21 |
| Sodium Benzoate | 0.25 | 0.15 | 0.18 | 0.25 | 0.15 | 0.18 | 0.25 |
| Disodium EDTA | 0.10 | 0.05 | 0.20 | 0.10 | 0.05 | 0.20 | 0.10 |
| PAD Microcapsule in form of PMC | 0.5 | 1.4 | 0.2 | 0.6 | 1.0 | — | — |
| PAD Matrix System | — | 0.3^{m} | 0.4 | — | 0.6 | — | 1.1 |
| Amine-Assisted Delivery | 0.2 | — | — | — | — | 0.4 | — |
| Pro-Perfume | — | — | — | — | 0.2^{q} | 0.6^{t} | — |
| Perfume-Loaded Tubules | — | — | — | — | — | — | 0.3 |
| Low KI Perfume Accord | — | — | — | 0.2 | 0.2 | 0.1 | 0.3 |
| Additional Perfume | 0.5 | 0.3 | 0.7 | 0.4 | 0.6 | 1.1 | — |
| Water/Carriers/Aesthetics* | | | | | | | |
| PAD Hot Melt** | — | Yes | — | Yes | Yes | — | — |
| PAD Perfumed Plastic** | — | — | Yes | — | — | — | Yes |

*balance
**Hot Melt and/or Perfumed Plastic on or in package not included in formula composition balance. For this example, FMOT technology is on or under the cap.
^{m}PAD Matrix System comprising a Silicone-Assisted Delivery (SAD) system
^{q}Pro-perfume comprising a nitrogen-based photo-pro-perfume (PPP) system that releases a coumarin derivative and a perfume aldehyde.
^{t}Pro-perfume comprising an ester-based photo-pro-perfume (PPP) system that releases a coumarin derivative and a perfume alcohol.

### Example #21

In one aspect of the present invention, the perfume delivery system is particular useful for antiperspirant/deodorant. In another aspect, the perfume delivery system consists of:

| Ingredients | | | | | | | |
|---|---|---|---|---|---|---|---|
| (% by weight) | A | B | C | D | E | F | G |
| Cyclomethicone | 7 | 7 | 6 | 8 | 7 | 7 | 7 |
| Dimethicone copolyol | 5 | 5 | 5 | 6 | 5 | 5 | 5 |
| Polydimethylsiloxane | 7 | — | 3 | 6 | 7 | 6 | 8 |
| Aluminium | 50 | 53 | 51 | 50 | 50 | 51 | 49 |
| hydrocholoride | | | | | | | |
| Propylene glycol | 15 | 15 | 15 | 15 | 15 | 15 | 15 |
| PAD Reservoir System | 0.6 | 1.2 | 0.8 | 0.5 | 1.5 | 2.4 | 0.2 |
| PAD Matrix System | — | 1.8 | 0.4 | — | — | — | 1.1 |
| Amine-Assisted | 0.2 | — | — | — | — | 0.4 | — |
| Delivery | | | | | | | |
| Cyclodextrin | — | — | — | 1.4 | 0.7 | — | 2.2 |
| Starch Encapsulated | — | — | 0.6 | — | — | — | — |
| Accord | | | | | | | |
| Low ODT Perfume | — | — | — | 0.2 | 0.2 | 0.1 | 0.3 |
| Accord | | | | | | | |
| Additional Perfume | 0.5 | 0.3 | 0.7 | 0.4 | 0.6 | 1.1 | — |
| Water/Carriers/ | | | | | | | |
| Aesthetics* | | | | | | | |
| PAD Hot Melt | Yes | — | — | — | — | — | — |
| PAD Perfumed Plastic | — | — | Yes | — | — | — | — |

*Balance

| Ingredients (% by weight) | H | I | J | K | L | M | N |
|---|---|---|---|---|---|---|---|
| Cyclomethicone | 7 | 7 | 6 | 8 | 7 | 7 | 7 |
| Dimethicone copolyol | 5 | 5 | 5 | 6 | 5 | 5 | 5 |
| Polydimethylsiloxane | 7 | — | 3 | 6 | 7 | 6 | 8 |
| Aluminium hydrocholoride | 50 | 53 | 51 | 50 | 50 | 51 | 49 |
| Propylene glycol | 15 | 15 | 15 | 15 | 15 | 15 | 15 |
| PAD Reservoir System in form of PMC | — | — | — | — | 1.0 | — | — |
| Molecule Assisted Delivery | — | — | 3.5 | — | — | — | — |
| Cyclodextrin | 0.9 | 1.6 | — | 0.6 | — | 1.5 | — |
| Starch Encapsulated Accord | — | — | — | 0.4 | — | — | 0.6 |
| ZIC in form of PLZ | — | — | — | — | — | — | 0.4 |
| ZIC in form of PLT | — | 1.5 | — | — | — | — | — |
| Pro-Perfume | — | — | — | — | 0.2 | 0.4 | — |
| Low KI Perfume Accord | — | — | 0.5 | 0.4 | 0.2 | 0.1 | 0.3 |
| Additional Perfume | 1.5 | 0.2 | 2.3 | 1.4 | 0.6 | 1.1 | — |
| Water/Carriers/Aesthetics* | | | | | | | |
| PAD Hot Melt | — | Yes | — | Yes | — | — | Yes |
| PAD Perfumed Plastic | Yes | — | — | — | — | — | Yes |

*Balance

### Example #22

In one aspect of the present invention, the perfume delivery system is particular useful for Invisible Solid Antiperspirant Sticks. In another aspect, the perfume delivery system consists of:

| Ingredients (% by weight) | A | B | C | D | E | F | G |
|---|---|---|---|---|---|---|---|
| Aluminum Zirconium Tetrachlorhydrate | 25.3 | 25.3 | 25.3 | 25.3 | 25.3 | 25.3 | 25.3 |
| glycine¹ | | | | | | | |
| Cyclopentasiloxane | QS | QS | QS | QS | QS | QS | QS |
| Petrolatum | 5.0 | 4.7 | 4.7 | 5.0 | 4.7 | 4.7 | 4.7 |
| Ozokerite | 9.0 | 1.0 | 1.0 | 9.0 | 1.0 | 1.0 | 1.0 |
| Stearyl Alcohol | — | 12.0 | 12.0 | — | 12.0 | 12.0 | 12.0 |
| PPG-14 Butyl Ether | 4.0 | — | — | 4.0 | — | — | — |
| Castor Wax | 9.0 | 9.0 | 9.0 | 9.0 | 9.0 | 9.0 | 9.0 |
| Talc | — | 4.0 | 4.0 | — | 4.0 | 4.0 | 4.0 |
| Behenyl Alcohol | — | 0.2 | 0.2 | — | 0.2 | 0.2 | 0.2 |
| d-Panthenyl Triacetate | — | 1.0 | 1.0 | — | 1.0 | 1.0 | 1.0 |
| PAD Reservoir System in form of PMC | 0.4 | — | 0.4 | — | 1.0 | — | — |
| Molecule Assisted Delivery | — | — | 3.5 | — | 1.0 | — | — |
| Starch Encapsulated Accord | — | — | — | 0.4 | — | — | 0.6 |
| Perfume-Loaded Zeolite (PLZ) | — | 0.4 | — | — | — | — | — |
| Perfume-Loaded Tubules (PLT) | — | — | — | — | — | — | 1.5 |
| Pro-Perfume | — | — | — | — | 0.2 | 0.4 | — |
| Low KI Perfume Accord | — | — | 0.2 | 0.4 | 0.2 | 0.1 | 0.3 |
| Primary Fragrance | 0.75 | 0.70 | 0.80 | 0.72 | 0.71 | 0.78 | 1.25 |
| Secondary Fragrance High Impact | 0.50 | 0.45 | 0.55 | 0.80 | 0.30 | 0.44 | 0.30 |
| Accord in beta-cyclodextrin Complex² | | | | | | | |
| Additional Uncomplexed Cyclodextrin | 0.9 | 1.6 | — | 0.6 | — | 1.5 | — |
| Water/Carriers/Aesthetics* | | | | | | | |
| PAD Hot Melt | — | Yes | — | Yes | — | — | Yes |
| PAD Perfumed Plastic | Yes | — | — | — | — | — | Yes |

*Balance

### Example #23

In one aspect of the present invention, the perfume delivery system is particular useful for Cream Antiperspirant Sticks. In another aspect, the perfume delivery system consists of:

| Ingredients (% by weight) | A | B | C | D | E | F | G |
|---|---|---|---|---|---|---|---|
| Aluminum Zirconium Tetrachlorhydrate | 25.3 | 25.6 | 25.1 | 25.9 | 26.3 | 26.0 | 25.0 |
| glycine¹ | | | | | | | |
| Cyclopentasiloxane | 0.01 | 1.1 | — | — | 5 | trace | trace |
| Dimethicone | 5.2 | 5.1 | 4.9 | 4.5 | 5.5 | 4.9 | 5.0 |
| Syncrowax HGLC | 1.2 | 1.3 | 1.1 | 1.0 | 1.4 | 1.2 | 1.2 |
| Fully Hydrogenated High Erucic Acid | 4.0 | 5.0 | 6.0 | 5.5 | 5.2 | 6.2 | 5.0 |
| Rapeseed Oil | | | | | | | |
| Mineral Oil | 1.0 | — | 1.2 | — | 0.8 | — | — |
| PAD Reservoir System in form of PMC | 0.6 | 1.2 | 0.8 | 0.5 | 1.5 | 2.4 | 0.2 |
| PAD Matrix System | 1.8^{m} | — | 0.4 | — | — | 0.2 | 1.1 |
| Amine-Assisted Delivery | 0.2 | 0.3 | — | — | — | 0.4 | — |
| Starch Encapsulated Accord | — | — | — | — | — | — | 0.6 |
| Pro-Perfume | — | — | — | — | 0.2 | 0.4 | — |
| Primary Fragrance | 0.75 | 0.70 | 0.80 | — | 0.33 | 0.78 | 1.25 |
| Secondary Fragrance High Impact | 0.50 | 0.45 | 0.55 | 0.80 | 0.30 | 0.44 | 0.30 |
| Accord in beta-cyclodextrin Complex² | | | | | | | |
| Additional Uncomplexed Cyclodextrin | — | — | 0.2 | — | 1.7 | — | 0.3 |
| Water/Carriers/Aesthetics* | | | | | | | |
| PAD Hot Melt** | — | Yes | — | Yes | — | — | Yes |
| PAD Perfumed Plastic** | Yes | — | — | — | — | — | Yes |

*Balance
¹Metal to Chloride metal ratio = 1.25; 75% anhydrous unbuffered active level (Westwood Chemical Co.)
²Fragrance as described in US 2006/0263313
^{m}PAD Matrix System comprising a Silicone-Assisted Delivery (SAD) system

### Example #24

In one aspect of the present invention, the perfume delivery system is particular useful for a fragrance oil/fine fragrance composition. In another aspect, the perfume delivery system consists of:

| Ingredients (% by weight) | A | B | C | D | E | F | G |
|---|---|---|---|---|---|---|---|
| Fragrance³ | 1.5 | 5 | 4 | 7 | 12 | 5 | 4 |
| Cyclodextrin (CD)⁴ | 2.5 | 5 | — | — | 6 | — | — |
| Additional Alternative Cyclodextrin | — | 0.3 | — | — | — | — | — |
| PAD Reservoir System | — | — | 1.1 | — | 1.2 | — | 0.8 |
| PAD Matrix System | — | — | 2.1 | 1.8^{m} | — | — | — |
| Molecule Assisted Delivery | — | — | — | 2 | — | — | — |
| Amine-Assisted Delivery | 0.3 | — | — | — | 0.3 | 0.5 | 0.2 |
| Pro-Perfume | — | 0.4 | — | — | — | 0.7 | — |
| Deionized Water | 15.8 | 12.9 | 13.7 | 12.9 | — | 13.3 | 12.9 |
| Ethanol | to 100 | to 100 | to 100 | to 100 | to 100 | to 100 | to 100 |

^{m}PAD Matrix System comprising a Silicone-Assisted Delivery (SAD) system

| Ingredients (% by weight) | H | I | J | K | L | M | N |
|---|---|---|---|---|---|---|---|
| Fragrance³ | 1.5 | 5 | 4 | 7 | 12 | 5 | 4 |
| Cyclodextrin (CD)⁴ | 2.5 | — | — | 5 | — | — | — |
| PAD Reservoir System | — | — | 1.1 | — | — | 1.2 | 1.8 |
| PAD Matrix System in form of SAD | — | — | — | 1.8 | to 100 | to 100 | — |
| Molecule Assisted Delivery | — | — | — | — | — | — | to 100 |
| Amine-Assisted Delivery | — | — | — | — | — | — | — |
| Pro-Perfume in form of PPP | — | 0.4 | — | — | — | — | — |
| ZIC | 0.2 | — | — | — | — | — | — |
| Deionized Water | 15.8 | 12.9 | 13.7 | 12.9 | — | — | — |
| Ethanol | to 100 | to 100 | to 100 | to 100 | — | — | — |
| PAD Hot Melt | — | — | — | Yes | Yes | — | — |
| PAD Perfumed Plastic | — | Yes | — | — | — | — | Yes |

³Fragrance as selected from examples I-V in US 2007/0037731 A1.
⁴Beta W7 M available from Wacker-Chemie GmbH, Hanns-Seidel-Platz 4, Munchen, DE.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A packaged product comprising:
a.) packaging, said packaging optionally comprising, based on total packaging weight, from about 0.001% to about 10% of a free perfume and/or from about 0.001% to about 50% of a perfume packaging delivery system; and
b.) a product matrix comprising, based on total product matrix weight, from about 0.001% to about 30% of a free perfume and/or from about 0.001% to about 60% of a perfume delivery system.

2. A packaged product according to claim 1 wherein the ratio of free perfume to perfume delivery system for the package is from about 1:100 to about 20:1 and the ratio of free perfume to perfume delivery system for the product matrix is from about 1:1000 to about 20:1.

3. A packaged product according to claim 1 wherein said perfume packaging delivery system comprises a material selected from the group consisting of a hot melt delivery system, a perfume loaded plastic and mixtures thereof; and said product matrix comprises a perfuming material selected from the group consisting of free perfume, polymer assisted delivery, molecule-assisted delivery, amine-assisted delivery, cyclodextrin, starch encapsulated accord, zeolite/inorganic carrier system, a pro-perfume system and mixtures thereof.

4. A packaged product according to claim 3 wherein said packaging comprises a perfume packaging delivery system selected from the group consisting of a hot melt delivery system, a perfume loaded plastic and mixtures thereof; and said product matrix comprises a perfuming material selected from the group consisting of free perfume, perfume microcapsule, cyclodextrin, starch encapsulated accord, water-triggered pro-perfume, nanolatex, silicone-assisted delivery, polymeric amine-assisted delivery, encapsulated or coated perfume loaded zeolites, perfume-loaded tubules, amine reaction product, photo-pro-perfume and mixtures thereof.

5. A packaged product according to claim 1, wherein said product matrix is a fluid and said product matrix comprises, based on total product matrix weight, from about 0.001% to about 30% of a free perfume and/or from about 0.001% to about 30% of one or more perfume delivery systems.

6. A packaged product according to claim 1, wherein said product matrix comprises, based on total product matrix weight, from about 2% to about 99.9% water and a perfuming material selected from the group consisting of free perfume, polymer-assisted delivery, amine-assisted delivery, pro-perfume, molecule-assisted delivery, zeolite/inorganic carrier system and mixtures thereof.

7. A packaged product according to claim 1, wherein said product matrix comprises a perfuming material selected from the group consisting of a free perfume that comprises a perfume having a Kovats Index of less than about 1500, cyclodextrin, starch encapsulated accords, water-triggered pro-perfumes, microcapsules, perfume-loaded zeolites, perfume loaded tubules, amine-reaction products, photo-pro-perfumes delivery system and mixtures thereof.

8. A packaged product according to claim 7, wherein said product matrix comprises, based on total product matrix weight, 0.0001% to about 60% of said perfume having a Kovats Index of less than about 1500.

9. A packaged product according to claim 1, wherein said product matrix comprises a non-aqueous fluid and a perfuming material selected from the group consisting of a free perfume, cyclodextrin, starch encapsulated accords, polymer-assisted delivery, pro-perfumes, polymer assisted delivery, molecule-assisted delivery, amine assisted delivery, zeolite/inorganic carrier delivery system and mixtures thereof.

10. A packaged product according to claim 1, wherein said packaging comprises a perfume packaging delivery system selected from the group consisting of a hot melt delivery system, a perfume loaded plastic and mixtures thereof; and said product matrix comprises based on total product matrix weight, from about 2% to about 99.9% of a non-aqueous fluid and a perfuming material selected from the group consisting of a free perfume having PRMs with Kovats Index values of less than about 1500, cyclodextrin, starch encapsulated accords, water-triggered pro-perfumes, microcapsules, perfume-loaded zeolites, perfume loaded tubules, amine-reaction products, photo-pro-perfumes delivery system, and mixtures thereof.

11. A packaged product according to claim 10, wherein said product matrix comprises, based on total product matrix weight, 0.0001% to about 60% of said perfume having a Kovats Index of less than about 1500.

12. A packaged product according to claim 1, wherein said product matrix comprises a solid said product matrix comprising, based on total product matrix weight, from about 0.001% to about 30% of a free perfume and/or from about 0.001% to about 30% of one or more perfume delivery systems.

13. A packaged product according to claim 12, wherein said packaging comprises a perfume packaging delivery system selected from the group consisting of a hot melt delivery system, a perfume loaded plastic and mixtures thereof; and said product matrix comprises a solid, said product matrix comprising a perfuming material selected from the group consisting of free perfume, cyclodextrin, starch encapsulated accords, polymer-assisted delivery, pro-perfumes, molecule-assisted delivery, amine assisted delivery, a zeolite/inorganic carrier delivery system and mixtures thereof.

14. A packaged product according to claim 13, wherein said product matrix comprises a solid, said product matrix comprising a perfuming material selected from the group consisting of a free perfume having PRMs with Kovats Index values of less than about 1500, cyclodextrin, starch encapsulated accords, water-triggered pro-perfumes, microcapsules, perfume-loaded zeolites, perfume loaded tubules, amine-reaction products, photo-pro-perfumes, and mixtures thereof.

15. A packaged product according to claim 12, wherein said product matrix comprises, based on total product matrix weight, 0.0001% to about 60% of said perfume having a Kovats Index of less than about 1500.

16. A packaged product according to claim 1, comprising an apparatus packaged by said packaging, said packaging comprising a perfume packaging delivery system selected from the group consisting of free perfume, one or more perfume delivery systems and mixtures thereof; and said apparatus comprising, based on total apparatus weight, from about 0.001% to about 30% of a free perfume and/or from about 0.001% to about 30% of a perfume delivery system.

17. A packaged product according to claim 1, comprising an apparatus packaged by said packaging, said packaging comprising a perfume packaging delivery system selected from the group consisting of a free perfume, cyclodextrin, starch encapsulated accords, polymer-assisted delivery, pro-perfumes, molecule-assisted delivery, amine assisted delivery, zeolite/inorganic carrier delivery systems and mixtures thereof.

18. A packaged product according to claim 17, wherein said apparatus comprises a perfuming material selected from the group consisting of free perfume, cyclodextrin, starch encapsulated accords, microcapsules, perfume-loaded zeolites, perfume loaded tubules, amine-reaction products, photo-pro-perfumes and mixtures thereof.

19. A packaged product according to claim 1 comprising a perfume microcapsule and a matrix system and/or amine assisted delivery system.

20. A packaged product according to claim 1 comprising a pro-perfume and a perfume microcapsule, a matrix system and/or amine assisted delivery system.

21. A packaged product according to claim 20 comprising wherein said pro-perfume comprises a photo pro-perfume.

22. A packaged product according to claim 1 comprising nanotubules.

23. A packaged product according to claim 22 comprising a material selected from the group consisting of a polymer assisted delivery system, an amine assisted delivery system, an amine reaction product, cyclodextrin, a starch encapsulated accord, a perfume loaded zeolite, a coated perfume loaded zeolite, a pro-perfume and mixtures thereof.

24. A packaged product comprising:
a.) a product matrix comprising, based on total product matrix weight, from about 0.001% to about 30% of a free perfume and/or from about 0.001% to about 60% of a perfume delivery system; and
b.) optionally, packaging, said packaging comprising, based on total packaging weight, comprising from about 0.001% to about 10% free perfume and/or from about 0.001% to about 50% of a perfume packaging delivery system.
